(19) United States
(12) Patent Application Publication
(10) Pub. No.: US 2006/0058249 A1
(43) Pub. Date: Mar. 16, 2006

(54) Efficient nucleic acid encapsulation into medium sized liposomes

(76) Inventors: Shangguan Tong, Princeton, NJ (US); Xingong Li, Cranbury, NJ (US); Paul R Meers, Princeton, NJ (US); Walter R Perkins, Pennington, NJ (US)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 10/500,933

(22) PCT Filed: Jan. 8, 2003
(86) PCT No.: PCT/US03/00380

§ 371 Date: Sep. 14, 2005

Related U.S. Application Data
(60) Provisional application No. 60/346,284, filed on Jan. 9, 2002.

(51) Int. Cl. A61K 48/00; A61K 9/127

(52) U.S. Cl. 514/43; 514/44; 424/450

(57) ABSTRACT

A method for preparing liposomes containing at least one nucleic acid encapsulated therein comprising the following steps: (A) mixing a gel or a liquid containing gel particles with aqueous medium Z1 to directly form the liposomes containing the at least one nucleic acid encapsulated therein; (B)(i) mixing a gel or a liquid containing gel particles with aqueous medium Z1 to form a curd or curdy substance; and (ii) mixing the curd or curdy substance with aqueous medium Z2 to directly form the liposomes containing the at least one nucleic acid encapsulated therein; or (C)(i) cooling a gel or a liquid containing gel particles to form a waxy substance; and (ii) mixing the waxy substance with aqueous medium Z1 to directly form the liposomes containing the at least one nucleic acid encapsulated therein; wherein said gel or liquid containing gel particles comprises at least one liposome-forming lipid, at least one fusogenic lipid, a water-miscible organic solvent and the at least one nucleic acid; wherein an amount of the at least one fusogenic lipid is at least 20% by weight of a lipid content of the gel or the liquid containing gel particles; and wherein the aqueous media Z1 and Z2 are the same or different.

### FIELD OF THE INVENTION

This invention concerns a method of preparing liposomes containing a nucleic acid encapsulated therein, liposomes containing a nucleic acid encapsulated therein prepared by said method, and methods of using the liposomes containing the nucleic acid. The method of preparing the liposomes of the present invention has the advantages of being simple and able to generate primarily small liposomes of relatively homogeneous particle size with a high entrapment efficiency. The liposomes containing a plasmid DNA encapsulated therein are useful in transfection of cells with high transfection efficiencies.

### BACKGROUND OF THE INVENTION

Gene therapy involves the delivery of a gene of interest to inside the cells of a subject in need of the therapy. There are two major groups of gene delivery systems used in gene therapy: viral and nonviral delivery systems. Viral delivery systems, e.g., using adenoviruses or herpes simplex II viruses, are quite efficient, but the systems suffer disadvantages of toxicity, immunogenicity of the viral components, potential risk of reversion of the virus to a replication-competent state, potential introduction of tumorigenic mutations, lack of targeting mechanism, limitations in DNA capacity and difficulty in large-scale production. Non-viral delivery systems are cationic liposome-DNA complexes, i.e., lipoplexes, liposome containing a DNA encapsulated therein along with a DNA condensing agent, or polymer complexes, i.e., polyplexes (see Shangguan et al, Gene Therapy 7:769-783, 2000). These non-viral delivery systems protect the DNA from extracellular DNases by condensation (in lipoplexes and polyplexes) or physical separation of the DNA from the extracellular environment via a lipid bilayer (in true liposomes carrying the DNA). The true liposomes of the prior art carrying the DNA require the inclusion of a DNA condensing agent, e.g., polycations of charge 3+ or higher, such as polyamines. The method of the present invention prepares liposomes containing a nucleic acid encapsulated therein without any requirement of the DNA condensing agent. Thus, the present invention is related to the use of liposomes as carrier of the nucleic acid. The liposomes prepared by the method of the present invention are useful in gene therapy if the nucleic acid encapsulated is a DNA.

Liposomes are lipid vesicles having at least one aqueous phase completely enclosed by at least one lipid bilayer membrane. Liposomes can be unilamellar or multilamellar. Unilamellar liposomes are liposomes having a single lipid bilayer membrane. Multilamellar liposomes have more than one lipid bilayer membrane with each lipid bilayer membrane separated from the adjacent lipid bilayer membrane by an aqueous layer. The cross sectional view of multilamellar vesicles is often characterized by an onion-like structure.

Liposomes are known to be useful in drug delivery, so many studies have been conducted on the methods of liposome preparation. Descriptions of these methods can be found in numerous reviews (e.g., Szoka et al., “Liposomes: Preparation and Characterization”, in Liposomes: From Physical Structure to Therapeutic Applications, edited by Knight, pp. 51-82, 1981; Deamer et al., “Liposome Preparation: Methods and Mechanisms”, in Liposomes, edited by Ostro, pp. 27-51, 1987; Perkins, “Applications of Liposomes with High Captured Volume”, in Liposomes Rational Design, edited by Janoff, pp. 219-259, 1999).

A method of preparing multilamellar liposome was first reported by Bangham et al. (J. Mol. Biol. 13:238-252, 1965). In the method of Bangham et al., phospholipids were mixed with an organic solvent to form a solution. The solution was then evaporated to dryness leaving behind a film of phospholipids on the internal surface of a container. An aqueous medium is added to the container to form multilamellar vesicles (hereinafter referred to as MLVs).

Small unilamellar vesicles (hereinafter referred to as SUVs) were prepared using sonication (Huang, Biochemistry 8:346-352, 1969). A phospholipid was dissolved in an organic solvent to form a solution, which was dried under nitrogen to remove the solvent. An aqueous phase was added to produce a suspension of vesicles. The suspension was sonicated until a clear liquid was obtained, which contained a dispersion of SUVs.

Other methods for the preparation of liposomes were discovered in the 1970s. These methods include the solvent-infusion method, the reverse-phase evaporation method and the detergent removal method. In the solvent-infusion method, a solution of a phospholipid in an organic solvent, most commonly ethanol, was rapidly injected into a larger volume of an aqueous phase under a condition that caused the organic solvent to evaporate. When the organic solvent evaporated upon entry into the aqueous phase, bubbles of the organic solvent's vapor were formed and the phospholipid was left as a thin film at the interface of the aqueous phase and the vapor bubble. As the vapor bubble ascended through the aqueous phase, the phospholipid spontaneously rearranged to form unilamellar and oligolamellar liposomes (e.g., see Batzri et al., Biochim. Biophys. Acta, 298:1015-1019, 1973). Liposomes produced by the solvent-infusion method were mostly unilamellar.

Large unilamellar vesicles (hereinafter referred to as LUVs) were prepared by the reverse-phase evaporation method. In the reverse-phase evaporation method, lipids were dissolved in an organic solvent, such as diethylether, to form a lipid solution. An aqueous phase was added directly into the lipid solution in a ratio of the aqueous phase to the organic solvent of 1:3 to 1:6. The mixture of the lipid/organic solvent/aqueous phase was briefly sonicated to form a homogenous emulsion of inverted micelles. The organic solvent was then removed from the mixture in a two-step procedure, in which the mixture was evaporated at 200-400 mm Hg until the emulsion became a gel, which was then evaporated at 700 mm Hg to remove all the solvent allowing the micelles to coalesce to form a homogeneous dispersion of mainly unilamellar vesicles known as reverse-phase evaporation vesicles (hereinafter referred to as REVs) (e.g., see Papahaduopoulos, U.S. Pat. No. 4,235,871).

In the detergent removal method, a phospholipid was dispersed with a detergent, such as cholate, deoxycholate or Triton X-100, in an aqueous phase to produce a turbid suspension. The suspension was sonicated to become clear as a result of the formation of mixed micelles. The detergent was removed by dialysis or gel filtration to obtain the liposomes in the form of mostly large unilamellar vesicles (e.g., see Enoch et al., Proc. Natl. Acad. Sci. USA, 76:145-149, 1979). The liposomes prepared by the detergent removal method suffer a major disadvantage in the inability to completely remove the detergent, with the residual detergent changing the properties of the lipid bilayer and affecting retention of the aqueous phase.

There were also methods for the preparation of large liposomes involving fusion or budding. These methods generally started with liposomes prepared with another method and disrupted the vesicular structures using mechanical or electrical forces. The disruption induced physical strain in the bilayer structure and changed the hydration and/or surface electrostatics. One of the ways of disrupting the existing vesicular structures was by a freezing and thawing process, which produced vesicle rupture and fusion. The freezing and thawing process increased the size and entrapment volume of the liposome.

Fountain et al. (U.S. Pat. No. 4,588,578) described a method for preparing monophasic lipid vesicles (hereinafter referred to as MPVs), which are lipid vesicles having a plurality of lipid bilayers. MPVs are different from MLVs, SUVs, LUVs and REVs. In the method of Fountain et al., a lipid or lipid mixture and an aqueous phase were added to a water-miscible organic solvent in amounts sufficient to form a monophase. The solvent was then evaporated to form a film. An appropriate amount of the aqueous phase was added to suspend the film, and the suspension was agitated to form the MPVs.

Minchey et al. (U.S. Pat. No. 5,415,867) described a modification of the method of Fountain et al. In the method of Minchey et al., a phospholipid, a water-miscible organic solvent, an aqueous phase and a biologically active agent were mixed to form a cloudy mixture. The solvents in the mixture were evaporated, but not to substantial dryness, under a stream of air in a warm water bath at 37° C. until the mixture formed a monophase, i.e., a clear liquid. As solvent removal continued, the mixture became opaque and gelatinous, in which the gel state indicated that the mixture was hydrated. The purging was continued for 5 minutes to further remove the organic solvent. The gelatinous material was briefly heated at 51° C. until the material liquified. The resulting liquid was centrifuged to form lipid vesicles containing the biologically active agent. The aqueous supernatant was removed and the pellet of lipid vesicles was washed several times. The modification of Minchey et al. was that the biologically active agent and the lipid were maintained as hydrated at all times to avoid the formation of a film of the biologically active agent and lipid upon the complete removal of all the aqueous phase. During evaporation of the organic solvent, the presence of a gel indicated that the monophase was hydrated.

Different techniques were developed to improve the encapsulation efficiency for nucleic acids. However, little progress has been made to conveniently and efficiently encapsulate molecules, especially large molecules such as DNA and RNA, into small or medium sized liposomes or to devise liposome production to make liposomes of a relatively homogeneous size distribution without resorting to size reduction methodologies (e.g. extrusion and homogenization). The prior art methods of preparing liposomes suffer from some or all of the following problems: being time consuming and not economical, having a low entrapment efficiency and/or generating vesicles of heterogenous size distribution requiring sonication or extrusion to remove large vesicles. An improved method of preparing liposomes containing a nucleic acid encapsulated therein is needed. The present invention solves the problems by presenting a new relatively simple method of making liposomes containing a nucleic acid encapsulated therein having a high entrapment efficiency and of relatively homogeneous size.

The method of the present invention is especially useful in encapsulating a plasmid DNA in liposomes. The liposomes so prepared using the gel hydration method of the present invention are useful in the transfection of eukaryotic cells due to their high transfection efficiency. As a result, the liposomes prepared by the method of the present invention are useful in gene therapy.

### SUMMARY OF THE INVENTION

The present invention involves the formation of liposomes via the hydration of a gel or a liquid containing gel particles, wherein the gel or the liquid containing gel particles comprise at least one liposome-forming lipid in a water-miscible organic solvent, preferably at a high concentration, and an aqueous medium, preferably in a small amount.

One of the aspects of the present invention concerns a method of preparing liposomes containing at least one nucleic acid encapsulated therein, said method comprising the following steps:
(A) mixing a gel or a liquid containing gel particles with aqueous medium Z1 to directly form the liposomes containing the at least one nucleic acid encapsulated therein, wherein said gel or liquid containing gel particles comprises at least one liposome-forming lipid, at least one fusogenic lipid, a water-miscible organic solvent and the at least one nucleic acid;
(B) (i) mixing a gel or a liquid containing gel particles with aqueous medium Z1 to form a curd or curdy substance, wherein said gel or liquid containing gel particles comprises at least one liposome-forming lipid, at least one fusogenic lipid, a water-miscible organic solvent and the at least one nucleic acid; and
   (ii) mixing the curd or curdy substance with aqueous medium Z2 to directly form the liposomes containing the at least one nucleic acid encapsulated therein,
(C) (i) cooling a gel or a liquid containing gel particles to form a waxy substance, wherein said gel or liquid containing gel particles comprises at least one liposome-forming lipid, at least one fusogenic lipid, a water-miscible organic solvent and the at least one nucleic acid; and
   (ii) mixing the waxy substance with aqueous medium Z1 to directly form the liposomes containing the at least one nucleic acid encapsulated therein;
(D) mixing a gel or a liquid containing gel particles with aqueous medium Z1 and the at least one nucleic acid to directly form the liposomes containing the at least one nucleic acid encapsulated therein, wherein said gel or liquid containing gel particles comprises at least one liposome-forming lipid, at least one fusogenic lipid and a water-miscible organic solvent;
(E) (i) mixing a gel or a liquid containing gel particles with aqueous medium Z1 and the at least one nucleic acid to form a curd or curdy substance, wherein said gel or liquid containing gel particles comprises at least one liposome-forming lipid, at least one fusogenic lipid and a water-miscible organic solvent; and
   (ii) mixing the curd or curdy substance with aqueous medium Z2 to directly form the liposomes containing the at least one nucleic acid encapsulated therein,
(F) (i) mixing a gel or a liquid containing gel particles with aqueous medium Z1 to form a curd or curdy substance, wherein said gel or liquid containing gel particles comprises at least one liposome-forming lipid, at least one fusogenic lipid and a water-miscible organic solvent; and
   (ii) mixing the curd or curdy substance with aqueous medium Z2 and the at least one nucleic acid to directly form the liposomes containing the at least one nucleic acid encapsulated therein;
(G) (i) cooling a gel or a liquid containing gel particles to form a waxy substance, wherein said gel or liquid containing gel particles comprises at least one liposome-forming lipid, at least one fusogenic lipid, a water-miscible organic solvent and the at least one nucleic acid; and
   (ii) mixing the waxy substance with aqueous medium Z1 to directly form the liposomes containing the at least one nucleic acid encapsulated therein; or
(H) (i) cooling a gel or a liquid containing gel particles to form a waxy substance, wherein said gel or liquid containing gel particles comprises at least one liposome-forming lipid, at least one fusogenic lipid and a water-miscible organic solvent; and
   (ii) mixing the waxy substance with aqueous medium Z1 and the at least one nucleic acid to directly form the liposomes containing the at least one nucleic acid encapsulated therein;
wherein the at least one liposome-forming lipid and the at least one fusogenic lipid are the same or different; and wherein the aqueous media Z1 and Z2 are the same or different.

In certain embodiments of the method of preparing the liposomes containing the nucleic acid encapsulated therein of the present invention, the amount of the at least one fusogenic lipid is at least about 20%, at least about 30%, at least about 40%, at least about 50%, at least about 60%, at least about 70%, at least about 75%, at least about 80%, at least about 85% or at least about 90% by weight of the lipid content of the gel or the liquid containing gel particles.

In certain embodiments of the method of preparing the liposomes containing the nucleic acid encapsulated therein of the present invention, the gel or the liquid containing gel particles can be prepared by a method comprising the following steps:
(I) (a) (aa) mixing the at least one liposome-forming lipid, the at least one fusogenic lipid, the at least one nucleic acid and the water-miscible 0.5 organic solvent to form a mixture; or
   (bb) (i) dissolving the at least one liposome-forming lipid and the at least one fusogenic lipid in the water-miscible organic solvent to form an organic solution;
      (ii) dissolving the at least one nucleic acid in aqueous medium X to form an aqueous solution; and
      (iii) mixing the organic solution and aqueous solution to form a mixture; or
   (b) mixing the at least one liposome-forming lipid, the at least one fusogenic lipid and the water-miscible organic solvent to form a mixture; and thereafter
(II) (a) mixing the mixture of step (I)(a) with aqueous medium Y to form the gel or liquid containing gel particles; or
   (b) mixing the mixture of step (I)(b) with the at least one nucleic acid and aqueous medium Y to form the gel or liquid containing gel particles,
wherein aqueous media X and Y are the same or different.

In certain embodiments of the method of preparing the liposomes containing the nucleic acid encapsulated therein starting with the preparation of the gel or the liquid containing gel particles, the gel or the liquid containing gel particles is formed without creation of any gas/aqueous phase boundary by sonication or any other method (the application of high frequency energy, wherein “high frequency energy” is energy having a frequency equal to at least the frequency of ultrasound).

In certain embodiments of the method of preparing the liposomes containing the nucleic acid encapsulated therein of the present invention, the gel or the liquid containing gel particles can be prepared by a method comprising the following steps:
(I) (a) (i) providing liposomes comprising the at least one liposome-forming lipid and the at least one fusogenic lipid, wherein the liposomes are prepared by a method other than the instant method; and
   (ii) mixing the liposomes of step (I)(a)(i) with the at least one nucleic acid;
   (b) (i) providing liposomes comprising the at least one liposome-forming lipid and the at least one fusogenic lipid in aqueous medium U, wherein the liposomes are prepared by a method other than the instant method; and
      (ii) mixing the liposomes of step (I)(b)(i) with the at least one nucleic acid;
   (c) (i) providing liposomes comprising the at least one liposome-forming lipid and the at least one fusogenic lipid, wherein the liposomes are prepared by a method other than the instant method; and
      (ii) mixing the liposomes of step (I)(c)(i) with aqueous medium U and the at least one nucleic acid;
   (d) (i) providing liposomes comprising the at least one liposome-forming lipid and the at least one fusogenic lipid in aqueous medium U, wherein the liposomes are prepared by a method other than the instant method; and
      (ii) mixing the liposomes of step (I)(d)(i) with aqueous medium U and the at least one nucleic acid; or
   (e) forming liposomes comprising the at least one liposome-forming lipid and the at least one fusogenic lipid in the presence of the at least one nucleic acid by a method other than the instant method;
(II) (a) mixing the product of step (I)(b), (I)(c) or (I)(d) with the water-miscible organic solvent to form the gel or the liquid containing gel particles; or
   (b) mixing the product of step (I)(a) or (I)(e) with aqueous medium V and the water-miscible organic solvent to form the gel or the liquid containing gel particles,
wherein aqueous media U and V are the same or different.

Within the scope of the present invention are liposomes containing the at least one nucleic acid encapsulated therein as prepared by any of the above preparation methods.

The present invention is also directed toward methods of using the liposomes containing the at least one nucleic acid encapsulated therein as prepared by any of the above preparation methods in cell transfection, gene therapy, vaccination or diagnosis.

When the at least one nucleic acid encapsulated is a DNA, especially a plasmid DNA, the liposomes containing the at least one nucleic acid encapsulated therein are useful for transfection of cells.

### BRIEF DESCRIPTION OF THE DRAWINGS

FIG. 1 shows, under a light microscope (magnification 400×), N-C12-DOPE/DOPC (in a 70/30 molar ratio, with a volume ratio of aqueous phase:ethanol of 2:1) liposomes prepared according to the method of the present invention before (top panel) and after (bottom panel) extrusion through a membrane filter having a 0.4 μm pore size.

FIG. 2 depicts the appearance of N-C12-DOPE/DOPC (70/30) liposomes prepared according to the method of the present invention under freeze-fracture electron microscopy.

FIG. 3 depicts the appearance of N-C12-DOPE/DOPC (70/30) liposomes prepared according to the method of the present invention under cryo electron microscopy.

FIG. 4 shows the encapsulation efficiencies and particle sizes of N-C12-DOPE/DOPC (70/30) liposomes containing DNA prepared according to the method of the present invention. Three particle sizes were given for the samples in the order of: mean particle diameter weighted by number, mean particle diameter weighted by light reflection intensity and mean particle diameter weighted by volume. The particle sizes were below 400 nm. Also shown were the final DNA concentration, lipid concentration and ratio of DNA to lipid in the liposomes.

FIG. 5 shows the results of fractionation of N-C12-DOPE/DOPC liposomes prepared according to the method of the present invention in a 5-20% sucrose gradient. The lipids were homogeneously distributed with no phase separation. The liposomes in the peak fractions had entrapment of 2.1+/−0.2 μl/μmol of lipids. The open squares, labeled “p/pc”, represented the phosphate to choline molar ratios, as determined by the respective assays, of the fractions separated by the sucrose gradient.

FIG. 6 is the phase diagram of a lipids-ethanol-aqueous buffer system, wherein the lipids were N-C12-DOPE/DOPC (70/30, molar ratio). The three axes of the ternary phase diagram show the individual weight fractions of the three components (lipids, ethanol or aqueous buffer) based on the sum of the weight of the three components. In the region above line a, the mixture was a clear liquid. In the region between line a and line b, the mixture existed as a cloudly liquid. In the region between line b and line c, the mixture was in a clear gel state. In the region between line c and line d, the mixture existed as a cloudy gel. In the region below line d, the mixture became liposomes with the appearance of a cloudy liquid. Therefore, in the phase diagram, the region above line b was the fluid zone and the region below line d was the liposome zone with the intermediate region (between line b and line d) being the gel zone. A study showed that the presence of a EGFP plasmid DNA did not alter the lipids/ethanol/aqueous medium ternary phase diagram.

FIG. 7 shows the light scattering of 100 μg/ml enhanced green fluorescence protein (hereinafter referred to as EGFP) plasmid DNA in ethanol-LSB solution with or without 200 mM sodium chloride, wherein “LSB” represented “low salt buffer.” In the presence of 200 mM sodium chloride, the DNA started to aggregate at 30% (wt/wt) ethanol, while without 200 mM sodium chloride, the DNA started to aggregate at 55% (wt/wt) ethanol.

FIG. 8 shows the transfection of OVCAR-3 cells with N-C12-DOPE/DOPC (70/30) liposomes (washed to remove unencapsulated DNA) prepared by the gel-hydration method of the present invention using ethanol as the water-miscible organic solvent, wherein the liposomes (washed to remove unencapsulated DNA) contained EGFP plasmid DNA encapsulated therein. After incubation of the OVCAR-3 cells with the liposomes, the transfection activity was determined based on the expression of the EGFP plasmid DNA in the OVCAR-3 cells. The transfection activity did not require any plasmid DNA condensing agent or any extrusion, which was a liposome size reduction process.

FIG. 9 shows the transfection of OVCAR-3 cells with N-C12-DOPE/DOPC (70/30) liposomes (washed to remove unencapsulated DNA) prepared by the gel-hydration method of the present invention using ethanol as the water-miscible organic solvent, wherein the liposomes (washed to remove unencapsulated DNA) contained luciferase plasmid DNA encapsulated therein. After incubation of the OVCAR-3 cells with the liposomes, the transfection activity was determined based on the expression of the luciferase gene in the plasmid DNA in the OVCAR-3 cells. The liposomes could transfect the OVCAR-3 cells in the presence of 10% serum (FBS stands for fetal bovine serum) with or without targeting via transferrin.

FIG. 10 shows the transfection of OVCAR-3 cells with N-C12-DOPE/DOPC (70/30) liposomes prepared by the gel-hydration method of the present invention using ethanol as the water-miscible organic solvent, wherein the liposomes contained luciferase plasmid DNA encapsulated therein. After incubation of the OVCAR-3 cells with the liposomes at various concentrations of CaCl₂ and MgCl₂, the transfection activity was determined based on the expression of the luciferase gene in the plasmid DNA in the OVCAR-3 cells. The liposomes could transfect the OVCAR-3 cells at physiological Ca²⁺ and M²⁺ concentrations, i.e., about 1.2 mM Ca²⁺ and 0.8 mM M²⁺.

FIG. 11 shows the transferrin mediated binding of N-C12-DOPE/DOPC (70/30) liposomes prepared by the gel-hydration method of the present invention using ethanol as the water-miscible organic solvent (see Example 13). The binding experiment was conducted in the presence of 10% FBS.

FIG. 12 shows the transferrin mediated transfection of N-C12-DOPE/DOPC (70/30) liposomes prepared by the gel-hydration method of the present invention using ethanol as the water-miscible organic solvent, wherein the liposomes contained PGL-3 plasmid DNA encapsulated therein. The experiment was conducted in the presence of 10% FBS.

FIG. 13 shows the transfection activity of liposomes prepared with pure DOPC, DOPC/N-C12-DOPE (8:2 molar ratio), DOPC/N-C12-DOPE (6:4 molar ratio), DOPC/N-C12-DOPE (4:6 molar ratio), DOPC/N-C12-DOPE (2:8 molar ratio) or pure N-C12-DOPE using the gel hydration method of the present invention in OVCAR-3 cells in culture. After incubation of the cells with the liposomes, the expression of the EGFP gene in the cells was determined by measuring the intensity of green fluorescence.

FIG. 14 shows the encapsulation efficiencies, for dextran fluorophores, of N-C12-DOPE/DOPC (70/30) liposomes prepared using the gel hydration method of the present invention or using a process for making stable plurilamellar vesicles (SPLV). The N-C12-DOPE/DOPC liposomes prepared according to the gel-hydration method of the present invention had a much higher encapsulation efficiency than the N-C12-DOPE/DOPC liposomes prepared using the SPLV process.

### DETAILED DESCRIPTION OF THE INVENTION

The method of preparing liposomes containing a nucleic acid encapsulated therein of the present invention involves hydration of a mixture of at least one nucleic acid, at least one liposome-forming lipid, at least one fusogenic lipid and a water-miscible organic solvent in the form of a gel or a liquid containing gel particles. In the mixture of the at least one nucleic acid, the at least one liposome-forming lipid, at least one fusogenic lipid and the water-miscible organic solvent, the liposome-forming lipid and the fusogenic lipid are typically dissolved in the water-miscible organic solvent, preferably at high concentrations. The mixture is typically mixed with a small amount of an aqueous medium to form the gel or the liquid containing gel particles. Hydration of the gel or the liquid containing gel particles leads to direct formation of liposomes without any additional manipulation, such as evaporation or sonication, normally required in prior art methods. Depending on the liposome-forming lipid used, in the liposome preparation method of the present invention, upon hydration the gel or the liquid containing gel particles may go through a curd or curdy stage before forming liposomes, but no additional manipulation, such as evaporation or sonication, is required other than hydration of a curd or curdy substance if the intermediate curd or curdy substance is formed upon hydration of the gel or the liquid containing gel particles. For instance, when certain saturated liposome-forming lipids are used in the methods, the gel or gel particles go through the curd or curdy stage upon hydration before liposome formation. Alternatively, in the liposome preparation method of the present invention, the gel or the liquid containing gel particles can be cooled to form a waxy substance, and the waxy substance is hydrated to directly form the liposomes without requiring any additional manipulation, such as sonication or evaporation.

In certain embodiments of the method of preparing the liposomes containing the nucleic acid encapsulated therein of the present invention, the gel or the liquid containing gel particles is formed without using any hydrating agent. The hydrating agent is a compound having at least two ionizable groups, one of which ionizable groups is capable of forming an easily dissociative ionic salt, which salt can complex with the ionic functionality of the liposome-forming lipid. The hydrating agent inherently does not form liposomes in and of itself and the hydrating agent must also be physiologically acceptable. Preferably, the at least two ionizable groups of the hydrating agent are of opposite charge. Examples of the hydrating agent are arginine, homoarginine, γ-aminobutyric acid, glutamic acid, aspartic acid and similar amino acids.

In certain embodiments of the method of preparing the liposomes containing the nucleic acid encapsulated therein, the gel or liquid containing gel particles is formed without the creation of any gas/aqueous phase boundary. The gel or liquid containing gel particles is formed by mixing the at least one liposome-forming lipid, the water-miscible organic solvent and aqueous medium Y without sonication or any other method (such as the application of high frequency energy to the mixture of the at least one liposome-forming lipid, the water-miscible organic solvent and aqueous medium Y) of producing a gas/aqueous phase boundary. The “high frequency energy” is the energy having a frequency at least equal to the frequency of ultrasound.

In certain embodiments of the method of preparing the liposomes containing the nucleic acid encapsulated therein of the present invention, a phospholipid content of the gel or the liquid containing gel particles used in the method is not 15 to 30% by weight of the gel or the liquid containing gel particles.

In certain embodiments of the method of preparing the liposomes containing the nucleic acid encapsulated therein of the present invention, a phospholipid content of the gel or the liquid containing gel particles used in the method is not 15 to 30% by weight of the gel or the liquid containing gel particles and the content of the water-miscible organic solvent is not 14 to 20% by weight of the gel or the liquid containing gel particles.

In certain embodiments of the method of preparing the liposomes containing the nucleic acid encapsulated therein of the present invention, the gel or the liquid containing gel particles used in the method further comprises at least one acidic phospholipid, wherein two or all of the at least one phospholipid, the at least one liposome-forming lipid and the at least one fusogenic lipid are the same or different. The content of the at least one phospholipid in the gel or the liquid containing gel particles is from about 30% to about 100%, about 40% to about 100%, about 50% to about 100%, about 60% to about 100%, about 70% to about 100%, or about 80% to about 100% by weight of the lipid(s) of the gel or the liquid containing gel particles.

In certain embodiments of the method of preparing the liposomes containing the nucleic acid encapsulated therein of the present invention, the gel or the liquid containing gel particles used in the method further comprises at least one charged lipid, wherein two or all of the at least one charged lipid, the at least one liposome-forming lipid and the at least one fusogenic lipid are the same or different. The content of the at least one charged lipid in the gel or the liquid containing gel particles is from about 40% to about 100%, about 50% to about 100%, about 60% to about 100%, about 70% to about 100%, or about 80% to about 100% by weight of the lipid(s) of the gel or the liquid containing gel particles. One of the benefits of adding at least one charged lipid in forming the liposomes is that the liposomes formed would have a small size, i.e., a preferred mean diameter, weighted by number, of about 400 nm or less, about 300 nm or less, about 200 nm or less, or about 100 nm or less, without the requirement of any sonication to form the gel or liquid containing gel particles, or the requirement of any sonication or extrusion of the liposomes.

Within the scope of the method of preparing the liposomes containing the nucleic acid encapsulated therein of the present invention is an embodiment in which no nucleic acid condensing agent, e.g., a polycation of charge +3 or higher such as polylysine, polyamine and hexammine cobalt (III), is used.

In the method of preparing the liposomes containing the nucleic acid encapsulated therein of the present invention, “to directly form the liposomes” means that the liposomes are formed without requiring any additional procedure or manipulation, such as evaporation or sonication, other than going through a potential intermediate stage of formation of a curd or curdy substance if certain liposome-forming lipids are used or through formation of an intermediate waxy substance if the gel or the liquid containing gel particles is cooled. For instance, in the method of preparing the liposomes encapsulating the at least one nucleic acid, mixing the gel or the liquid containing gel particles comprising the at least one nucleic acid with aqueous medium Z1 leads directly to the formation of the liposomes having the at least one nucleic acid entrapped without the requirement of any additional procedure or manipulation, such as evaporation or sonication, other than the hydration of a curd or curdy intermediate if certain saturated liposome-forming lipids are used. Alternatively, if the gel or the liquid containing gel particles comprising the at least one nucleic acid is cooled to form a waxy substance, the hydration of the waxy substance leads directly to the formation of the liposomes having the at least one nucleic acid entrapped without the requirement of any additional procedure or manipulation, such as evaporation or sonication.

In the method of preparing the liposomes containing the nucleic acid encapsulated therein of the present invention, the aqueous medium X, aqueous medium Y, aqueous medium Z1 and/or aqueous medium Z2 is preferably an aqueous buffer. Examples of the aqueous buffer include citrate buffer, Tris buffer, phosphate buffer and a buffer containing sucrose or dextrose.

In the method of preparing the liposomes containing the nucleic acid encapsulated therein of the present invention, the gel or the liquid containing gel particles and aqueous medium Z1 are mixed by either adding aqueous medium Z1 to the gel or the liquid containing gel particles, or adding or infusing the gel or the liquid containing gel particles into aqueous medium Z1.

The at least one “liposome-forming lipid” is any lipid that is capable of forming liposomes. Typically, the at least one “liposome-forming lipid” is a lipid that can form lipid bilayers. Examples of the liposome-forming lipid include phospholipids, glycolipids and sphingolipids. The phospholipids that are liposome-forming include phosphatidylcholine, phosphatidylserine, phosphatidylinositol, phosphatidylglycerol, diphosphatidylglycerol and N-acyl phospatidylethanolamine. Examples of the liposome-forming phospholipid include phospholipids selected from the group consisting of dioleoyl phosphatidylcholine, dipalmitoyl phosphatidylcholine, distearoyl phosphatidylcholine, dimyristoyl phosphatidylcholine, 1-palmitoyl-2-oleoyl-sn-glycero-3-phosphocholine, 1-oleoyl-2-palmitoyl-sn-glycero-3-phosphocholine, 1,2-dioleoyl-sn-glycero-3-[phospho-rac-(1-glycerol)], 1,2-dipalmitoyl-sn-glycero-3-[phospho-rac-(1-glycerol)], 1,2-distearoyl-sn-glycero-3-[phospho-rac-(1-glycerol)], 1,2-dimyristoyl-sn-glycero-3-[phospho-rac-(1-glycerol)], 1-palmitoyl-2-oleoyl-sn-glycero-3-[phospho-rac-(1-glycerol)], 1-oleoyl-2-palmitoyl-sn-glycero-3-[phospho-rac-(1-glycerol)], N-decanoyl phosphatidylethanolamine, N-dodecanoyl phosphatidylethanolamine and N-tetradecanoyl phosphatidylethanolamine.

Preferably, the at least one liposome-forming lipid is phosphatidylcholine, e.g., dioleoyl phosphatidylcholine, dipalmitoyl phosphatidylcholine, distearoyl phosphatidylcholine, dimyristoyl phosphatidylcholine, 1-palmitoyl-2-oleoyl-sn-glycero-3-phosphocholine and 2-palmitoyl-1-oleoyl-sn-glycero-3-phosphocholine, or N-acyl phosphatidylethanolamine, e.g., 1,2-dioleoyl-sn-glycero-N-decanoyl-3-phosphoethanolamine, 1,2-dioleoyl-sn-glycero-N-dodecanoyl-3-phosphoethanolamine, 1,2-dioleoyl-sn-glycero-N-tetradecanoyl-3-phosphoethanolamine, 1,2-dipalmitoyl-sn-glycero-N-decanoyl-3-phosphoethanolamine, 1,2-dipalmitoyl-sn-glycero-N-dodecanoyl-3-phosphoethanolamine, 1,2-dipalmitoyl-sn-glycero-N-tetradecanoyl-3-phosphoethanolamine, 1-oleoyl-2-palmitoyl-sn-glycero-N-decanoyl-3-phosphoethanolamine, 1-oleoyl-2-palmitoyl-sn-glycero-N-dodecanoyl-3phosphoethanolamine, 1-oleoyl-2-palmitoyl-sn-glycero-N-tetradecanoyl-3-phosphoethanolamine, 1-palmitoyl-2-oleoyl-sn-glycero-N-decanoyl-3-phosphoethanolamine, 1-palmitoyl-2-oleoyl-sn-glycero-N-dodecanoyl-3-phosphoethanolamine, and 1-palmitoyl-2-oleoyl-sn-glycero-N-tetradecanoyl-3-phosphoethanolamine.

In method of preparing the liposomes containing the nucleic acid encapsulated therein of the present invention, the at least one “fusogenic lipid” is a lipid that, upon incorporation into a liposome, increases the fusogenicity of the liposome and examples of the “fusogenic lipid” include N-acyl phosphatidylethanolamine (see Meers et al, U.S. Pat. No. 6,120,797, the disclosure of which is herein incorporated by reference). The at least one liposome-forming lipid and the at least one fusogenic lipid are the same or different. Preferably, the at least one liposome-forming lipid is also a fusogenic lipid. For instance, when the at least one liposome-forming lipid is a N-acyl phosphatidylethanolamine, the N-acyl phosphatidylethanolamine is liposome-forming and also increases the fusogenicity of the liposomes (see U.S. Pat. No. 6,120,797). N-acyl phosphatidylethanolamine that can be used include N-decanoyl phosphatidylethanolamine, N-undecanoyl phosphatidylethanolamine, N-dodecanoyl phosphatidylethanolamine, N-tridecanoyl phosphatidylethanolamine, and N-tetradecanoyl phosphatidylethanolamine, e.g., 1,2-dioleoyl-sn-glycero-N-decanoyl-3-phosphoethanolamine, 1,2-dioleoyl-sn-glycero-N-dodecanoyl-3-phosphoethanolamine, 1,2-dioleoyl-sn-glycero-N-tetradecanoyl-3-phosphoethanolamine, 1,2-dipalmitoyl-sn-glycero-N-decanoyl-3-phosphoethanolamine, 1,2-dipalmitoyl-sn-glycero-N-dodecanoyl-3-phosphoethanolamine, 1,2-dipalmitoyl-sn-glycero-N-tetradecanoyl-3-phosphoethanolamine, 1-oleoyl-2-palmitoyl-sn-glycero-N-decanoyl-3-phosphoethanolamine, 1-oleoyl-2-palmitoyl-sn-glycero-N-dodecanoyl-3-phosphoethanolamine, 1-oleoyl-2-palmitoyl-sn-glycero-N-tetradecanoyl-3-phosphoethanolamine, 1-palmitoyl-2-oleoyl-sn-glycero-N-decanoyl-3phosphoethanolamine, 1-palmitoyl-2-oleoyl-sn-glycero-N-dodecanoyl-3-phosphoethanolamine, and 1-palmitoyl-2-oleoyl-sn-glycero-N-tetradecanoyl-3-phosphoethanolamine. The fusogenicity-increasing N-acyl phosphatidylethanolamine is preferably N-dodecanoyl phosphatidylethanolamine and more preferably 1,2-dioleoyl-sn-glycero-N-dodecanoyl-3-phosphoethanolamine.

The liposome prepared by the method of preparing liposomes containing the nucleic acid encapsulated therein of the present invention can further comprise a sterol. Preferably, the sterol is cholesterol. The sterol can be added during the formation of the gel or the liquid containing gel particles, or added to the gel or the liquid containing gel particles.

The liposomes prepared by the preparatory methods of the present invention can comprise one or a combination (at any ratio) of the following lipids (if a lipid is both liposome-forming and fusogenic, only one lipid is required but optionally at least one of the other lipids can be included in a combination; if a lipid is liposome-forming and not fusogenic, another lipid which is fusogenic is required but optionally at least one of the other lipids can be included in a combination; and if a lipid is fusogenic and not liposome-forming, another lipid which is liposome-forming is required but optionally at least one of the other lipids can be included in a combination): phosphatidylcholines, phosphatidylglycerols, phosphatidylserines, phosphatidylethanolamines, phosphatidylinositols, headgroup modified phospholipids, headgroup modified phosphatidylethanolamines, lyso-phospholipids, phosphocholines (ether linked lipids), phosphoglycerols (ether linked lipids), phosphoserines (ether linked lipids), phosphoethanolamines (ether linked lipids), sphingomyelins, sterols, such as cholesterol hemisuccinate, tocopherol hemisuccinate, ceramides, cationic lipids, monoacyl glycerol, diacyl glycerol, triacyl glycerol, fatty acids, fatty acid methyl esters, single-chain nonionic lipids, glycolipids, lipid-peptide conjugates and lipid-polymer conjugates. However, in certain embodiments of the method of preparing the liposomes encapsulating the nucleic acid of the present invention, no phosphatidylcholine is used. In the methods of preparing the liposomes having the nucleic acid encapsulated therein of the present invention, the lipids can be added when the gel or the liquid containing gel particles are mixed with aqueous medium Z1 (e.g., the lipids can be a part of the gel or the liquid containing gel particles, or the lipids can be mixed with the aqueous medium and the gel or the liquid containing gel particles) or added before the gel or the liquid containing gel particles is formed (e.g., the lipids can be mixed with the water-miscible organic solvent, or the lipids can be a part of the liposome formed by a method other than the method of the present invention).

In certain embodiments of the method of preparing liposomes encapsulating the nucleic acid of the present invention, at least one charged lipid is added in preparing the liposomes having the nucleic acid encapsulated therein. The at least one charged lipid can be added during the formation of the gel or the liquid containing gel particles. Thus, the gel or the liquid containing gel particles can comprise at least one charged lipid, at least one liposome-forming lipid, at least one fusogenic lipid, the water-miscible organic solvent and the at least one nucleic acid, wherein some or all of the at least one charged lipid, the at least one liposome-forming lipid and the at least one fusogenic lipid are the same or different. Alternatively, the at least one charged lipid is added to the gel or the liquid containing gel particles. The “charged lipid” is a lipid having a net negative or positive charge in the molecule. Examples of the charged lipid include N-acyl phosphatidylethanolamine, phosphatidylserine, phosphatidylinositol, phosphatidylglycerol, diphosphatidylglycerol (i.e., cardiolipin) and phosphatidic acid.

In the method of the present invention, the water-miscible organic solvent is an organic solvent that, when mixed with water, forms a homogeneous liquid, i.e., with one phase. The water-miscible organic solvent can be selected from the group consisting of acetaldehyde, acetone, acetonitrile, allyl alcohol, allylamine, 2-amino-1-butanol, 1-aminoethanol, 2-aminoethanol, 2-amino-2-ethyl-1,3-propanediol, 2-amino-2-methyl-1-propanol, 3-aminopentane, N-(3-aminopropyl)morpholine, benzylamine, bis(2-ethoxyethyl) ether, bis(2-hydroxyethyl) ether, bis(2-hydropropyl) ether, bis(2-methoxyethyl) ether, 2-bromoethanol, meso-2,3-butanediol, 2-(2-butoxyethoxy)-ethanol, butylamine, sec-butylamine, tert-butylamine, 4-butyrolacetone, 2-chloroethanol, 1-chloro-2-propanol, 2-cyanoethanol, 3-cyanopyridine, cyclohexylamine, diethylamine, diethylenetriamine, N,N-diethylformamide, 1,2-dihydroxy-4-methylbenzene, N,N-dimethylacetamide, N,N-dimethylformaide, 2,6-ethylmorpholine, 1,4-dioxane, 1,3-dioxolane, dipentaerythritol, ethanol, 2,3-epoxy-1-propanol, 2-ethoxyethanol, 2-(2-ethoxyethoxy)-ethanol, 2-(2-ethoxyethoxy)-ethyl acetate, ethylamine, 2-(ethylamino)ethanol, ethylene glycol, ethylene oxide, ethylenimine, ethyl(−)-lactate, N-ethylnorpholine, ethyl-2-pyridine-carboxylate, formamide, furfuryl alcohol, furfurylamine, glutaric dialdehyde, glycerol, hexamethylphosphor-amide, 2,5-hexanedione, hydroxyacetone, 2-hydroxyethyl-hydrazine, N-(2-hydroxyethyl)-morpholine, 4-hydroxy-4-methyl-2-pentanone, 5-hydroxy-2-pentanone, 2-hydroxypropionitrile, 3-hydroxypropionitrile, 1-(2-hydroxy-1-propoxy)-2-propanol, isobutylamine, isopropylamine, 2-isopropylamino-ethanol, 2-mercaptoethanol, methanol, 3-methoxy-1-butanol, 2-methoxyethanol, 2-(2-methoxyethoxy)-ethanol, 1-methoxy-2-propanol, 2-(methylamino)-ethanol, 1-methylbutylamine, methylhydrazine, methyl hydroperoxide, 2-methylpyridine, 3-methylpyridine, 4-methylpyridine, N-methylpyrrolidine, N-methyl-2-pyrrolidinone, morpholine, nicotine, piperidine, 1,2-propanediol, 1,3-propanediol, 1-propanol, 2-propanol, propylamine, propyleneimine, 2-propyn-1-ol, pyridine, pyrimidine, pyrrolidine, 2-pyrrolidinone and quinoxaline. Acetonitrile, C₁-C₃ alcohols and acetone are preferred examples of the water-miscible organic solvent. The C₁-C3 alcohols are preferably methanol, ethanol, 1-propanol, 2-propanol, ethylene glycol and propylene glycol, and more preferably ethanol, 1-propanol or 2-propanol, with ethanol being the most preferred. One of the advantages of the method of the present invention is that an organic solvent, such as ethanol or acetone, of relatively low toxicity can be used. With a water-miscible organic solvent of relatively low toxicity, the liposomes prepared according to the method of the present invention would not be expected to pose any significant toxicity threat even when the liposomes contain a residual amount of the water-miscible organic solvent.

In the method of preparing liposomes containing the at least one nucleic acid encapsulated therein of the present invention, the total amount of the at least one liposome-forming lipid and the at least one fusogenic lipid in the gel or the liquid containing gel particles before the gel or liquid containing gel particles are mixed with aqueous medium Z1 can range from about 1% by weight of the gel or the liquid containing gel particles to the sum of the hydration limit of the at least one liposome-forming lipid and the hydration limit of the at least one fusogenic lipid in water. The “hydration limit” of a lipid is the maximum amount of the lipid in a given amount of water that would keep the lipid in a liposomal state. The total amount of the at least one liposome-forming lipid and the at least one fusogenic lipid in the gel or the liquid containing gel particles before the mixing with the aqueous medium Z1 can have a lower limit of about 5%, about 10%, about 15%, about 20%, about 30%, about 40%, about 50%, about 60% or about 70% by weight of the gel or the liquid containing gel particles before the gel or the liquid is mixed with the aqueous medium Z1, and an upper limit of about 95% by weight of the gel or the liquid containing gel particles before the gel or the liquid is mixed with the aqueous medium Z1. The total amount of the at least one liposome-forming lipid and the at least one fusogenic lipid in the gel or the liquid containing gel particles before the mixing with the aqueous medium Z1 can have a lower limit of about 5%, about 10%, about 15%, about 20%, about 30%, about 40%, about 50%, about 60% or about 70% by weight of the gel or the liquid containing gel particles before the gel or the liquid is mixed with the aqueous medium Z1, and an upper limit of about 90% by weight of the gel or the liquid containing gel particles before the gel or the liquid is mixed with the aqueous medium Z1. The total amount of the at least one liposome-forming lipid and the at least one fusogenic lipid in the gel or the liquid containing gel particles before the mixing with the aqueous medium Z1 can have a lower limit of about 5%, about 10%, about 15%, about 20%, about 30%, about 40%, about 50%, about 60% or about 70% by weight of the gel or the liquid containing gel particles before the gel or the liquid is mixed with the aqueous medium Z1, and an upper limit of about 85% by weight of the gel or the liquid containing gel particles before the gel or the liquid is mixed with the aqueous medium Z1. The total amount of the at least one liposome-forming lipid and the at least one fusogenic lipid in the gel or the liquid containing gel particles before the mixing with the aqueous medium Z1 can also be from about 5% to about 80%, about 10% to about 80%, about 15% to about 80%, about 20% to about 80%, about 30% to about 80%, about 40% to about 80%, about 50% to about 80%, about 60% to about 80%, about 70% to about 80%, about 10% to about 70%, about 20% to about 60%, or about 30% to about 50% by weight of the gel or the liquid containing gel particles before the gel or the liquid is mixed with the aqueous medium Z1. Alternatively, the total amount of the at least one liposome-forming lipid and the at least one fusogenic lipid in the gel or the liquid containing gel particles before the mixing with the aqueous medium Z1 ranges from about 60% to about 90%, or is about 45%, by weight of gel or the liquid containing gel particles.

In the method of preparing the liposomes containing the at least one nucleic acid encapsulated therein of the present invention, aqueous medium Z1 is preferably mixed with the gel or the liquid containing gel particles in increments. Mixing in increments has the advantage of yielding a higher entrapment efficiency compared with mixing the entire amount of aqeuous medium Z1 with the gel or the liquid containing gel particles in one step. The size of the increment can be up to about 1000%, up to about 500%, up to about 200%, up to about 100%, up to about 90%, up to about 80%, up to about 70%, up to about 60%, up to about 50%, up to about 40%, up to about 30%, up to about 20%, up to about 10%, up to about 5%, up to about 2%, up to about 1%, up to about 0.5%, up to about 0.1%, up to about 0.05% or up to about 0.01% of the weight of the gel or the liquid containing gel particles before the gel or the liquid is mixed with any aqueous medium Z1. The size of the increment can also be from about 0.001% to about 10%, from about 0.001% to about 5%, from about 0.001% to about 1% or from about 0.001% to about 0.1% of the weight of the gel or the liquid containing gel particles before the gel or the liquid is mixed with any aqueous medium Z1.

FIG. 6 shows the phase diagram of a lipids/water-miscible organic solvent/aqueous medium system that can be used in the liposome preparatory method of the present invention, wherein the lipids are N-C12-DOPE/DOPC (70/30, molar ratio). Ethanol was the water-miscible organic solvent and Tris buffer was the aqueous medium. The three axes of the ternary phase diagrams show the individual weight fractions of the three components (lipids, ethanol or aqueous buffer). In the ternary phase diagram, the liquid or solution zone, the gel zone and the liposome zone are depicted. Similar ternary phase diagrams can be generated by a person skilled in the art without undue experimentation for other lipid(s)/water-miscible organic solvent/aqueous medium systems. The method of the present invention can, however, be practiced without the ternary phase diagrams. The ternary phase diagrams are merely used herein to show the general relationship between the fluid zone, gel zone and liposome zone for the lipid(s)/water-miscible organic solvent/aqueous medium systems used in the methods of the present invention.

In one of the embodiments of the method of preparing liposomes of the present invention, after the liposomes are formed, the liposomes are washed with an aqueous medium by centrifugation, gel filtration or dialysis.

Liposomes are useful as delivery vehicles of encapsulated substances. The method of the present invention can be used to encapsulate at least one nucleic acid in liposomes. The liposomes containing the at least one nucleic acid encapsulated therein prepared by the method of the present invention have the advantages of a high entrapment efficiency and a relatively homogeneous particle size. Due to the simplicity of the procedures, the method of preparing the liposomes of the present invention allows relatively rapid production of the liposomes at a low cost. The method of the present invention has the additional advantage of being easily controlled and modified, e.g., by selecting a batch or continuous operation, to fit the special requirements of different formulations.

The at least one nucleic acid encapsulated in the liposomes of the present invention can be an oligonucleotide, RNA or DNA. The oligonucleotide that can be encapsulated can be of about 5 to about 500 bases in size. Examples of RNA that can be encapsulated in the liposomes prepared according to the present invention are anti-sense RNA and RNA interference, i.e., RNAᵢ.

The DNA that can be encapsulated in the liposomes prepared according to the present invention includes a plasmid DNA. The plasmid DNA can be of up to 20 kb, up to 15 kb, up to 10 kb, from about 0.5 kb to about 20 kb, from about 1 kb to about 15 kb, from about 2 kb to about 10 kb or from about 3 kb to about 7 kb in size. Liposomes of the present invention containing the plasmid DNA are useful in gene therapy, transfection of eukaryotic cells and transformation of prokaryotic cells. It was discovered that the liposomes prepared by the method of the present invention containing a plasmid DNA encapsulated therein have a high transfection efficiency.

The liposomes of the present invention having at least one nucleic acid encapsulated therein can be administered to a subject in need of the nucleic acid via an oral or parenteral route (e.g., intravenous, intramuscular, intraperitoneal, subcutaneous and intrathecal routes) for therapeutic or diagnostic purposes. The dose of the liposomes to be administered is dependent on the nucleic acid involved, and can be adjusted by a person skilled in the art based on the health of the subject and the medical condition to be treated or diagnosed. For diagnostic purposes, some the liposomes of the present invention can be used in vitro.

Within the scope of the present invention is a method of preventing or treating a health disorder in a subject in need of the treatment or prevention, said method comprises administering the liposomes containing at least one nucleic acid encapsulated therein as prepared by one of the above methods in the subject, wherein the at least one nucleic acid has the desired therapeutic or disease-preventing effect. The at least one nucleic acid can be an RNA, such as anti-sense RNA or RNAᵢ, or plasmid DNA.

Additionally, the present invention encompasses a method of transfecting cells with a DNA, said method comprises using the liposomes containing a DNA encapsulated therein by mixing the liposomes prepared according to the liposome preparatory method of the present invention with the cells with optional incubation. The DNA preferably is a plasmid DNA. The plasmid DNA preferably contains a gene of interest for the transfection.

Therefore, the liposomes prepared by the method of the present invention containing the plasmid DNA are useful in gene therapy, transfection of eukaryotic cells and transformation of prokaryotic cells. An aspect of the invention is a method for transfecting cells, preferably mammalian cells such as human cells, said method comprising contacting the cells in vivo or in vitro with the liposomes containing the plasmid DNA encapsulated therein as prepared by the method of the present invention, wherein the plasmid DNA preferably contains a gene of interest. The transfection method is also useful in a method for gene therapy comprising contacting target cells of a subject in need of the gene therapy with the liposomes containing the plasmid DNA encapsulated therein, in vitro (e.g., via incubation) or in vivo (e.g., via administration of the liposomes into the subject), wherein the plasmid DNA contains a gene having the desired therapeutic effect on the subject. Within the scope of the invention is a method of transforming prokaryotic cells comprising contacting (e.g., via incubation) the prokaryotic cells with the liposomes containing a plasmid DNA encapsulated therein as prepared by the method of the present invention to obtain transformation of the prokaryotic cells.

In the gel or the liquid containing gel particles used in the method of preparing liposomes containing the nucleic acid encapsulated therein of the present invention, a concentration of the nucleic acid can be up to about 40 mg/ml, up to about 30 mg/ml, up to about 20 mg/ml, up to about 10 mg/ml or up to about 5 mg/ml.

The liposomes containing the nucleic acid encapsulated therein prepared by the method of the present invention can further comprise a targeting agent to facilitate the delivery of the nucleic acid to a proper target in a biological system. Examples of the targeting agent include antibodies, a molecule containing biotin, a molecule containing streptavidin, or a molecule containing a folate or transferrin molecule.

Some aspects of the present invention are shown in the following working examples. However, the scope of the present invention is not to be limited by the working examples. A person skilled in the art can practice the present invention as recited in the claims beyond the breadth of the working examples. The working examples are included for illustration purposes only.

The names of certain chemicals used in the working examples were abbreviated as shown below:
1,2-dioleoyl-sn-glycero-3-phosphoethanolamine-N-dodecanoyl (N-C12-DOPE);
1,2-dioleoyl-sn-glycero-3-phosphocholine (DOPC);
1-palmitoyl-2-oleoyl-sn-glycero-3-phosphocholine (POPC);
1-palmitoyl-2-oleoyl-sn-glycero-3-[phospho-rac-(1-glycerol)] (POPG);
1,2-distearoyl-sn-glycero-3-phosphocholine (DSPC);
1,2-distearoyl-sn-glycero-3-[phospho-rac-(1-glycerol)] (DSPG) and enhanced green fluorescence protein plasmid DNA (EGFP plasmid DNA).

### EXAMPLE 1

N-C12-DOPE/DOPC Liposome Preparation by Ethanol Gel Hydration

Typically, 36.7 mg of N-C12-DOPE and 14.2 mg of DOPC were co-dissolved in 100 μl ethanol. A volume of 100-200 μl of an aqueous solution containing a biological active substance was injected into the lipid ethanol solution under intense mixing. Then 1.8 ml of a hydration buffer (300 mM sucrose, 10 mM Tris, 1 mM NaCl, pH 7.0) was slowly added to the sample to form a suspension of liposomes. Any unencapsulated material was removed by washing (one wash consisted of (1) sedimenting the liposomes in an aqueous phase, (2) replacing the supernatant with fresh aqueous phase, and (3) resuspending the pellet) the liposomes three times via 10,000 g centrifugation.

If the nucleic acid to be encapsulated was a EGFP plasmid DNA or PGL-3 plasmid, and the liposome-forming lipid to be used was a mixture of N-C12-DOPE/DOPC (in a molar ratio of 70/30), generally the following procedure could be used to prepare the liposomes with gel hydration. The lipid mixture, N-C12-DOPE/DOPC (in a molar ratio of 70/30), was dissolved in ethanol at a concentration of about 600 mM. The plasmid DNA was added in an aqueous solution at a concentration of about 1 to 4 mg/ml to the lipid ethanol solution to form a clear gel. The gel was hydrated by adding an aqueous buffer (10 mM Tris, 1 mM sodium chloride, 300 mM sucrose, pH 7.0) under intense mixing. The gel turned cloudy and finally collapsed after additional aqueous solution was added. The so formed liposome suspension was washed by centrifugation to remove any free plasmid DNA.

### EXAMPLE 2

Light Microscopy of N-C12-DOPE/DOPC Liposomes Prepared by Ethanol Gel Hydration

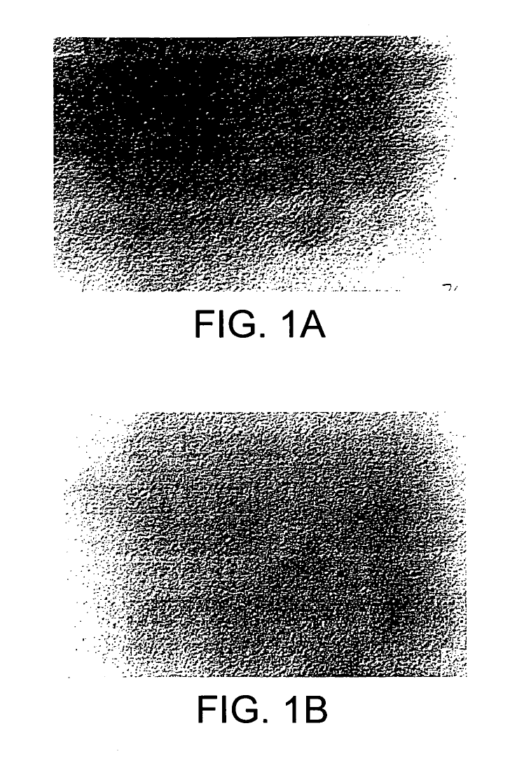

N-C12-DOPE/DOPC liposomes (70:30, molar ratio) were prepared by the gel hydration process (as set forth in Example 1) using 36.7 mg of N-C12-DOPE, 14.2 mg of DOPC and 400 μg of EGFP plasmid DNA. Light micrographs (Olympus BH-2, New York/New Jersey Scientific) of these liposomes before and after five passes of extrusion through a membrane filter with 400 nm pore size were taken at a magnification of 400× (see FIG. 1, top and bottom panels).

### EXAMPLE 3

Freeze Fracture Electron Microscopy of N-C12-DOPE/DOPC Liposomes Prepared by Ethanol Gel Hydration

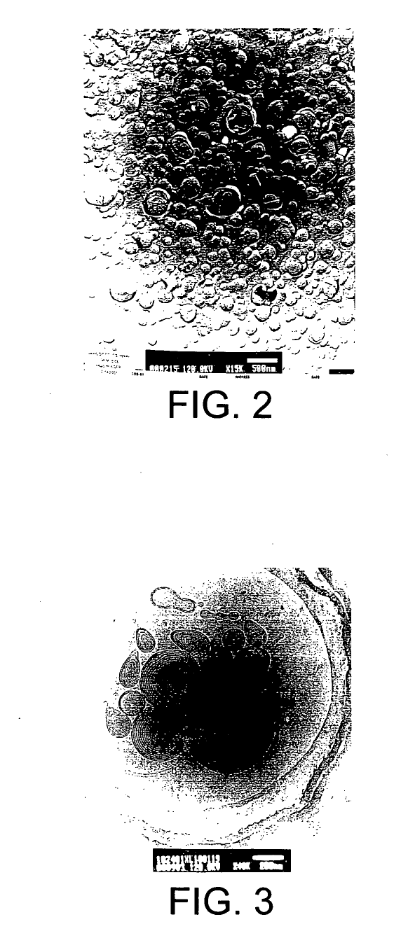

N-C12-DOPE/DOPC liposomes (70:30, molar ratio) were prepared by the gel hydration process (as set forth in Example 1) using 36.7 mg of N-C12-DOPE, 14.2 mg of DOPC and 400 μg PGL-3 plasmid DNA (a commercially available plasmid DNA containing luciferase as a reporter gene). Freeze fracture electron replicas were made and observed at magnifications of about 43,000× (see FIG. 2).

### EXAMPLE 4

Cryo Electron Microscopy of N-C12-DOPE/DOPC Liposomes Prepared by Ethanol Gel Hydration

N-C12-DOPE/DOPC liposomes (70:30, molar ratio) were prepared by the gel hydration process (as set forth in Example 1) using 36.7 mg of N-C12-DOPE, 14.2 mg of DOPC and 400 μg of EGFP plasmid DNA. Liposomes samples were placed on Quantifoil^{ó} 2/2 grids, blotted with a filtering paper to form a uniform thin film of liquid 1-2 mm in thickness, and flush-frozen by plunging into liquid ethane. Frozen samples were transferred to a Gatan 910 cryo-holder and observed at a magnification of 30,000× at an accelerating voltage of 120 kV in a Jeol JEM-1200EX electron microscope (FIG. 3).

### EXAMPLE 5

Particle Size Analysis

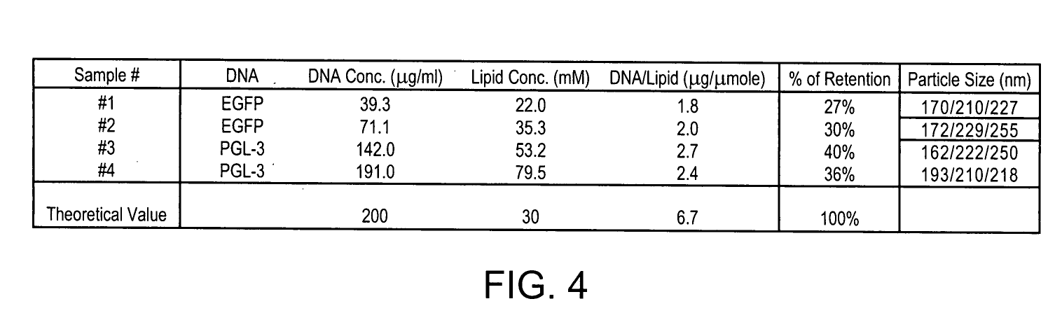

N-C12-DOPE/DOPC liposomes (70:30, molar ratio) were prepared by the gel hydration process (as set forth in Example 1) using 36.7 mg of N-C12-DOPE, 14.2 mg of DOPC and 400 μg PGL-3 or EGFP plasmid DNA. Their particle sizes were measure by a Submicron Particle Sizer (model 370), from NICOMP Particle Sizing Systems, Inc. Mean particle diameters (nm), as weighted by number, intensity or volume, were smaller than 400 nm (FIG. 4).

### EXAMPLE 6

DNA to Lipid Ratio Measurement

N-C12-DOPE/DOPC liposomes (70:30, molar ratio) were prepared by the gel hydration process (as set forth in Example 1) using 36.7 mg of N-C12-DOPE, 14.2 mg of DOPC and 400 μg PGL-3 or EGFP plasmid DNA. The liposomes had DNA:lipid ratios of about 1-2 μg/μmole (FIG. 4), as determined by a phosphate assay and Picogreen assay (Shangguan et al., Gene Therapy, 769-783, 2000), respectively. The plasmid DNA was protected against DNase I digestion as described in Shangguan et al.

### EXAMPLE 7

Sucrose Gradient Fractions of N-C12-DOPE/DOPC Liposomes Prepared by Ethanol Gel Hydration

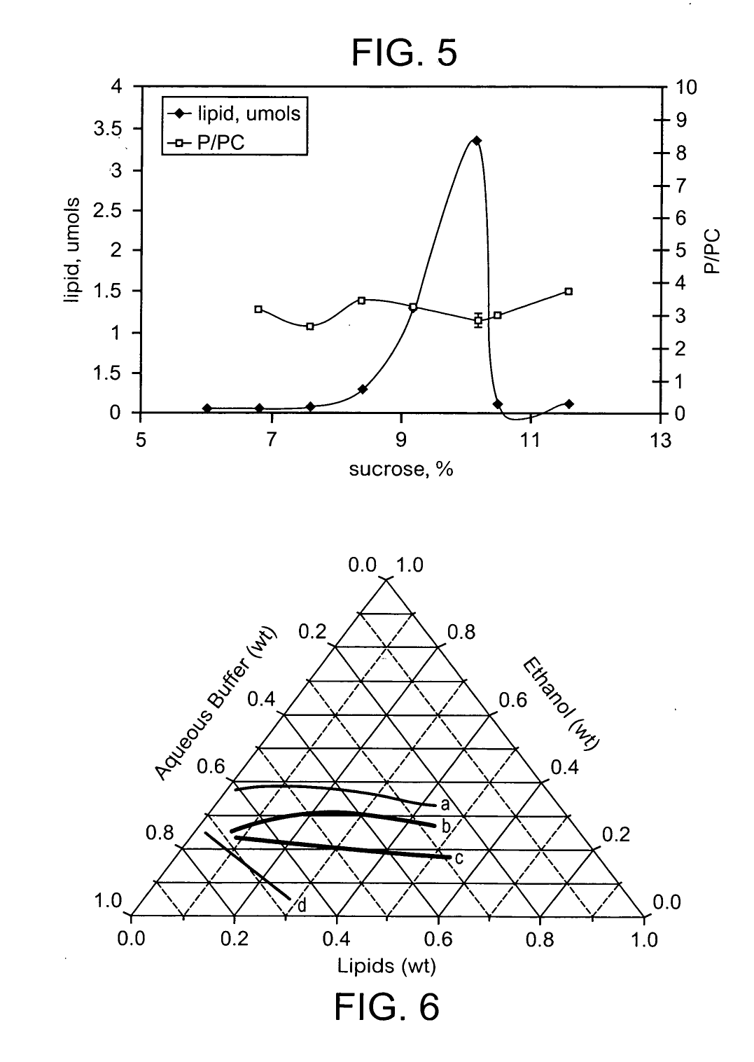

A 5-20% continuous sucrose gradient was obtained by mixing a 10 mM Tris buffer, pH 7, containing 140 mM NaCl, and a 10 mM Tris buffer, pH 7, containing 20% sucrose instead of NaCl. The liposomes were loaded on top of the gradient and centrifuged for 17 hours at 35,000 rpm. The centrifugation yielded a single band of liposomes centered at approximately 10% sucrose. The contents of the centrifuge tubes were fractionated starting from the bottom. The concentrations of the total phosholipids and DOPC were determined using phosphate and choline assays. In all fractions examined, the phosphate to choline ratios were nearly the same: 3±0.2 (see FIG. 5), which indicates compositional homogeneity of mixed lipid liposomes.

### EXAMPLE 8

N-C12-DOPE/DOPC—Ethanol—Aqueous Phase Diagram

Different amounts of 5-60 mg of N-C12-DOPE/DOPC lipid mixtures (70:30, molar ratio) were dissolved in 38-190 mg ethanol to reach lipid concentrations of 3%, 14%, 18%. 25%, 31%, 40%, and 60% (wt/wt). A 5 mM HEPES buffer (pH 7.5) was added incrementally to the lipid solutions at increments of 20-25 mg under intense mixing. The total weight of added buffer was recorded each time when the mixtures underwent a phase change. Similarly, 25.5-60 mg of N-C12-DOPE/DOPC lipid mixtures (70:30, molar ratio) were suspended in 34-77 mg of a 5 mM HEPES buffer (pH 7.5) to reach lipid concentrations of 25%, 33%, 43%, and 60% (wt/wt). Ethanol was added incrementally to the lipid suspensions at increments of 15-30 mg under intense mixing. The total weight of added ethanol was recorded each time when the mixtures underwent a phase change. A ternary lipids—ethanol—aqueous phase diagram was constructed by connecting the critical points at which the mixture underwent any phase change (FIG. 6).

### EXAMPLE 9

DNA Light Scattering in Ethanol Solutions.

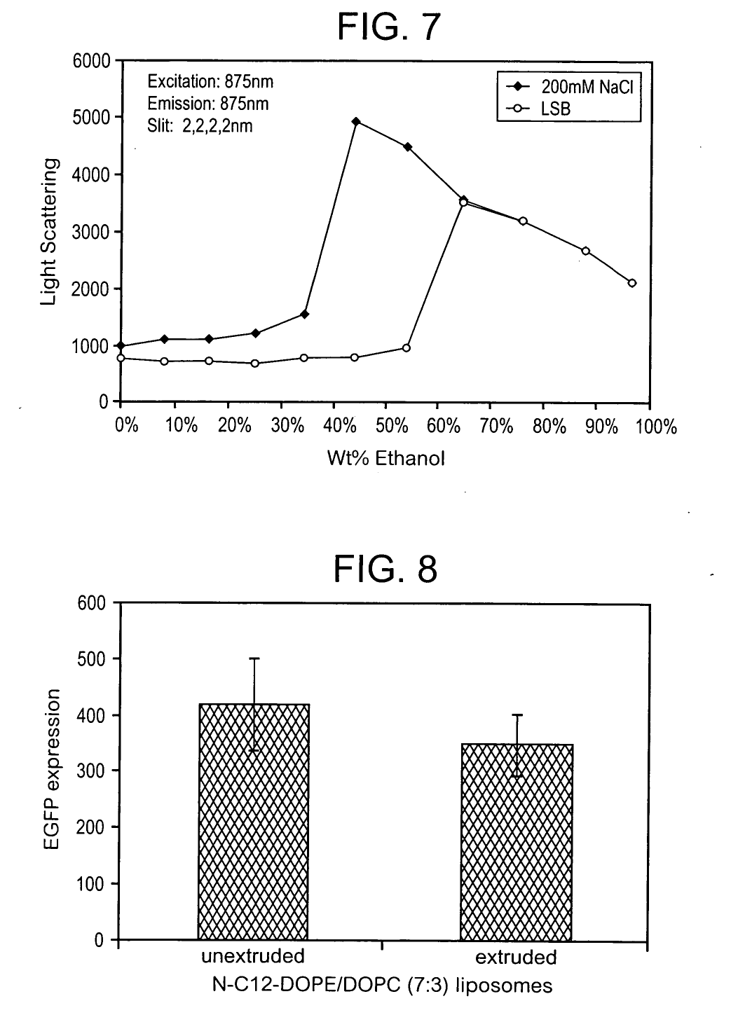

A volume of 85.7 μl of a EGFP plasmid DNA stock solution (3.5 mg EGFP plasmid DNA/ml) was added to each of 0-97% (wt/wt) ethanol solutions. In another experiment, the ethanol solution contained 200 mM NaCl. 90° light scattering of the EGFP plasmid DNA at 875 nm in different ethanol solutions was presented in FIG. 7. This experiment was conducted to determine the effect of ethanol on the plasmid DNA. The 200 mM NaCl solution was used to mimic the ionic strength in the gel containing N-C12-DOPE.

### EXAMPLE 10

Transfection Activity of N-C12-DOPE/DOPC (70:30) Liposomes Made by the Gel Hydration Method (FIG. 8)

The N-C12-DOPE/DOPC (70:30) liposomes containing the EGFP plasmid DNA were made by the gel hydration method as set forth in Example 1. Half of the sample was extruded through a 400 nm filter five times before removal of unencapsulated DNA. For a transfection assay, OVCAR3 cells were plated in 96 well plates at 2×10⁵ cells/ml in 0.1 ml/well of RPMI 1640 with 10% heat inactivated fetal bovine serum (FBS). The cells were allowed to grow for approximately 40-48 hours before transfections were performed. At this point the cells were at confluency. Transfection solutions (0.1 ml/well for 96 well plates) were prepared by dilution of appropriate liposome samples to approximately 2 mM total lipid (for equal lipid transfection) into medium with 0.5% FBS. The plates were aspirated to remove medium and washed once with Dulbecco's phosphate buffered saline (PBS) followed by aspiration. After an addition of a final concentration of 1 mM CaCl₂ and 0.4 mM MgCl₂, the transfection solution was then added to the wells and incubated at 37° C. for 3 hours. After incubation, the wells were aspirated and a medium containing 10% heat inactivated FBS was added to each well. Because of the previously demonstrated silencing of transgenes, 5 mM of a histone deacetylase inhibitor, butyrate, was added to each well to enhance expression. After incubation at 37° C. in a cell culture incubator for 18-22 hours, the medium was aspirated and a 0.1 ml wash of Dulbecco's PBS was added. For quantifying EGFP gene expression, samples were then dissolved in a detergent and readings were taken for corrected total EGFP fluorescence in terms of the total number of live cells as previously described (Shangguan et al., Gene Therapy, 769-783, 2000).

### EXAMPLE 11

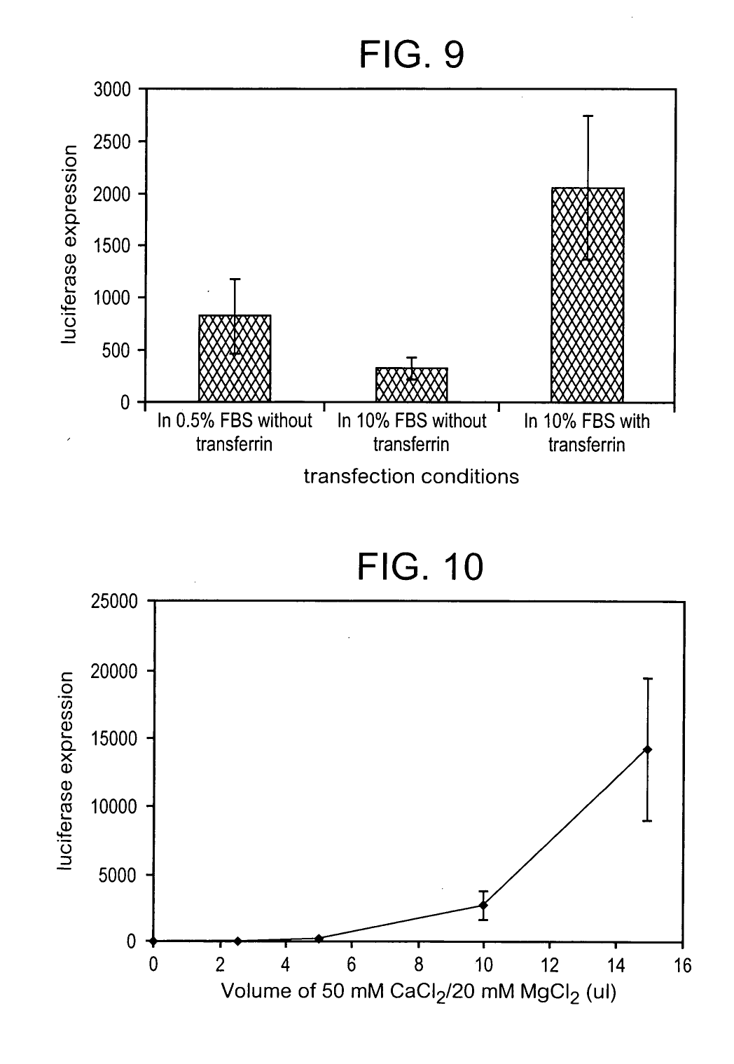

Transfection Activity of N-C12-DOPE/DOPC (70:30) Liposomes in the Presence of 10% Serum, with and without Targeting via Transferrin (FIG. 9)

The N-C12-DOPE/DOPC (70:30) liposomes containing PGL-3 plasmid were made by the gel hydration method as set forth in Example 1. Transfections without transferrin were performed as described in example 10, except that in one of the transfection assays, 10% FBS was used instead of 0.5% FBS. For transferrin targeted transfection, the liposome samples were first mixed with equal volumes of a 2 mg/ml poly-lysin transferrin conjugate at a concentration of 20 mM for 10 minutes, and then this mixture was diluted 10 times with Hank's balanced salt solution (HBSS) without Ca²⁺/Mg²⁺ containing 10% FBS before being applied to the cells. The level of luciferase expression was determined by the Bright-glow luciferase assay (Clontech).

In the presence of 0.5% FBS, without transferrin, the sample showed significant transfection activity. In the presence of 10% FBS, the sample showed decreased but still considerable transfection. In the presence of 10% FBS, with transferrin, the sample showed a dramatic increase of transfection activity (FIG. 9).

### EXAMPLE 12

Transfection Activity of N-C12-DOPE/DOPC (70:30) Liposomes at Physiological

Ca²⁺/Mg²⁺ Concentrations (FIG. 10)

The N-C12-DOPE/DOPC (70:30) liposomes containing PGL-3 plasmid were made by the gel hydration method as set forth in Example 1. The transfections were performed as described in example 10, in the presence of 0.5% FBS and without targeting, except that various volumes of CaCl₂ and MgCl₂ solution were added to 500 μl of the transfection solution before their addition to the cells at 100 μl per well to test the Ca²⁺/Mg²⁺ dependence of the transfection activity. The level of luciferase expression was determined by the Bright-glow luciferase assay (Clontech). The N-C12-DOPE/DOPC (70:30) liposomes had transfection activity at physiological concentrations of Ca²⁺-M²⁺, i.e., about 1.2 mM Ca²⁺ and 0.8 mM M²⁺ (FIG. 10).

### EXAMPLE 13

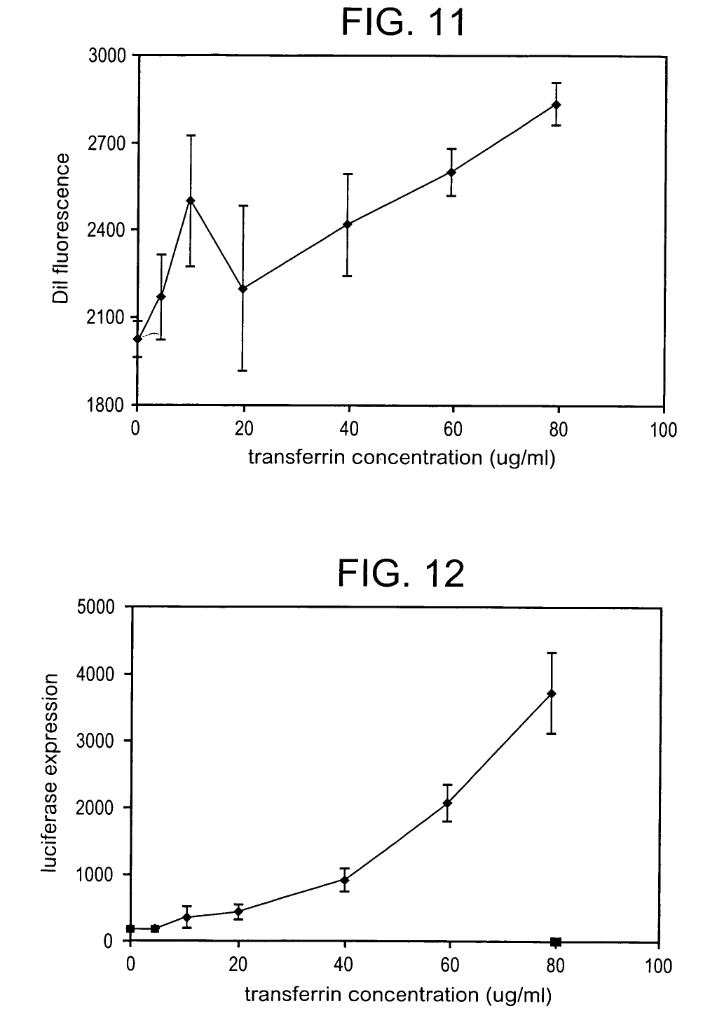

Transferrin Mediated Binding of N-C12-DOPE/DOPC (70:30) Liposomes in 10% FBS (FIG. 11)

The N-C12-DOPE/DOPC (70:30) liposomes containing fluorescent lipid probe Dil at a 0.1% (wt %) concentration were prepared by the ethanol gel hydration method as set forth in Example 1. The liposomes were incubated with OVCAR-3 cells in the presence of 10% FBS and various concentrations of transferrin as described in Example 11. After a 3 hour incubation at 37° C., the cells were washed three times with PBS and dissolved in 1% C12E8. Cell associated DiI fluorescence was measured at an emission wavelength of 620 nm, with an excitation wavelength of 560 nm. Binding of the liposome sample showed a small increase with increasing transferrin concentration (FIG. 11).

### EXAMPLE 14

Transferrin Mediated Transfection of N-C12-DOPE/DOPC (70:30) Liposomes in 10% FBS (FIG. 12)

The N-C12-DOPE/DOPC (70:30) liposomes containing PGL-3 plasmid were made by the gel hydration method as set forth in Example 1. The transfections were performed as described in Example 11, in the presence of 10% FBS and with various concentrations of transferrin for targeting. The level of luciferase expression was determined by the Bright-glow luciferase assay (Clontech). The liposome sample showed a transferrin dependent increase of transfection activity (FIG. 12).

### EXAMPLE 15

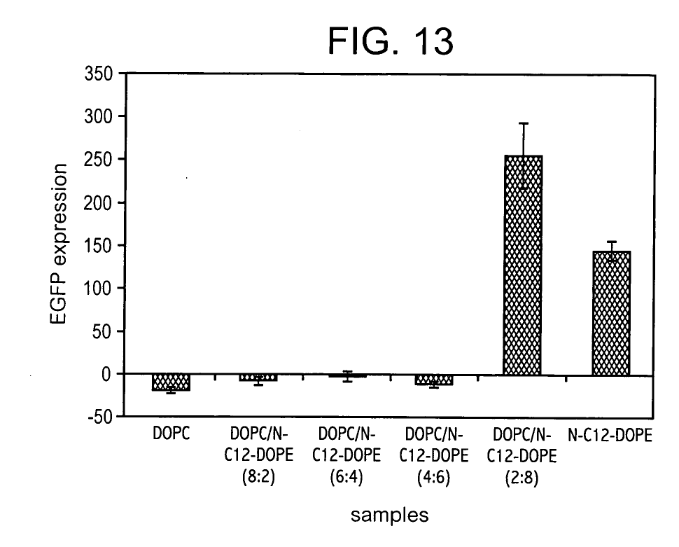

Transfection Activity of Liposomes Containing DOPC, N-C12-DOPE, or DOPC/N-C12-DOPE at Various Ratios (FIG. 13)

The liposomes containing a EGFP plasmid DNA and the following lipids or lipid mixtures, including 100% DOPC, DOPC/N-C12-DOPE (8:2 molar ratio), DOPC/N-C12-DOPE (6:4 molar ratio), DOPC/N-C12-DOPE (4:6 molar ratio), DOPC/N-C12-DOPE (2:8 molar ratio), and 100% N-C12-DOPE, were made by the ethanol gel hydration method as set forth in Example 1. The transfection assay was performed as described in Example 10.

### EXAMPLE 16

Encapsulation of Dextran

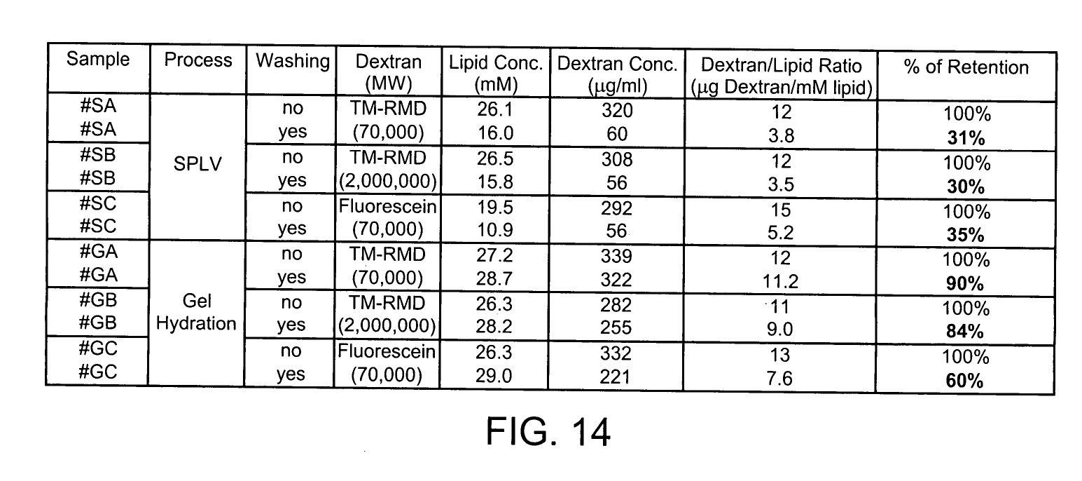

N-C12-DOPE/DOPC liposomes (70:30, molar ratio) were prepared by the gel hydration process (as set forth in Example 1) using 36.7 mg of N-C12-DOPE, 14.2 mg of DOPC and 100 μl of one of the following dextran stock solutions (5 mg/ml): tetramethyl rhodamine (MW 70,000), tetramethyl rhodamine (MW 2,000,000) or fluorescein (MW 70,000, lysine fixable). Conventional N-C12-DOPE/DOPC liposomes (70:30, molar ratio) were also prepared by the SPLV method: 1.13 ml of N-C12-DOPE/DOPC lipid mixtures (60 mM total lipid, 70:30 molar ratio) in chloroform were mixed with 100 μl of one of the following dextran stock solutions (5 mg/ml): tetramethyl rhodamine (MW 70,000), tetramethyl rhodamine (MW 2,000,000) or fluorescein (MW 70,000, lysine fixable). The mixture was sonicated briefly to form an emulsion. After most of the chloroform was removed by rotary evaporation at room temperature, 1.9 ml of a hydration buffer was added to the mixtures followed by additional 15 min of rotary evaporation. The unencapsulated material was removed by washing the liposomes three times via 10,000 g centrifugation. The dextran and lipid contents of each sample (FIG. 14) were determined using fluorescent measurement (excitation: 555 nm, emission: 580 nm) and a phosphate assay.

What is claimed is:

1. A liposome containing at least one nucleic acid encapsulated therein prepared according to a method comprising the following steps:
(A) mixing a gel or a liquid containing gel particles with aqueous medium Z1 to directly form the liposomes containing the at least one nucleic acid encapsulated therein, wherein said gel or liquid containing gel particles comprises at least one liposome-forming lipid, at least one fusogenic lipid, a water-miscible organic solvent and the at least one nucleic acid;
(B) (i) mixing a gel or a liquid containing gel particles with aqueous medium Z1 to form a curd or curdy substance, wherein said gel or liquid containing gel particles comprises at least one liposome-forming lipid, at least one fusogenic lipid, a water-miscible organic solvent and the at least one nucleic acid; and
(ii) mixing the curd or curdy substance with aqueous medium Z2 to directly form the liposomes containing the at least one nucleic acid encapsulated therein,
(C) (i) cooling a gel or a liquid containing gel particles to form a waxy substance, wherein said gel or liquid containing gel particles comprises at least one liposome-forming lipid, at least one fusogenic lipid, a water-miscible organic solvent and the at least one nucleic acid; and
(ii) mixing the waxy substance with aqueous medium Z1 to directly form the liposomes containing the at least one nucleic acid encapsulated therein;
(D) mixing a gel or a liquid containing gel particles with aqueous medium Z1 and the at least one nucleic acid to directly form the liposomes containing the at least one nucleic acid encapsulated therein, wherein said gel or liquid containing gel particles comprises at least one liposome-forming lipid, at least one fusogenic lipid and a water-miscible organic solvent;
(E) (i) mixing a gel or a liquid containing gel particles with aqueous medium Z1 and the at least one nucleic acid to form a curd or curdy substance, wherein said gel or liquid containing gel particles comprises at least one liposome-forming lipid, at least one fusogenic lipid and a water-miscible organic solvent; and
(ii) mixing the curd or curdy substance with aqueous medium Z2 to directly form the liposomes containing the at least one nucleic acid encapsulated therein,
(F) (i) mixing a gel or a liquid containing gel particles with aqueous medium Z1 to form a curd or curdy substance, wherein said gel or liquid containing gel particles comprises at least one liposome-forming lipid, at least one fusogenic lipid and a water-miscible organic solvent; and
(ii) mixing the curd or curdy substance with aqueous medium Z2 and the at least one nucleic acid to directly form the liposomes containing the at least one nucleic acid encapsulated therein;
(G) (i) cooling a gel or a liquid containing gel particles to form a waxy substance, wherein said gel or liquid containing gel particles comprises at least one liposome-forming lipid, at least one fusogenic lipid, a water-miscible organic solvent and the at least one nucleic acid; and
(ii) mixing the waxy substance with aqueous medium Z1 to directly form the liposomes containing the at least one nucleic acid encapsulated therein; or
(H) (i) cooling a gel or a liquid containing gel particles to form a waxy substance, wherein said gel or liquid containing gel particles comprises at least one liposome-forming lipid, at least one fusogenic lipid and a water-miscible organic solvent; and
(ii) mixing the waxy substance with aqueous medium Z1 and the at least one nucleic acid to directly form the liposomes containing the at least one nucleic acid encapsulated therein;
wherein the at least one liposome-forming lipid and the at least one fusogenic lipid are the same or different; and wherein the aqueous media Z1 and Z2 are the same or different and the amount of the at least one fusogenic lipid in the gel or the liquid containing gel particles is at least about 30% by weight of the lipid content of the gel or the liquid containing gel particles.

2. The liposome of claim 1, wherein the amount of the at least one fusogenic lipid is at least about 40% by weight of the lipid content of the gel or the liquid containing gel particles.

3. The liposome of claim 2, wherein the amount of the at least one fusogenic lipid is at least about 50% by weight of the lipid content of the gel or the liquid containing gel particles.

4. The liposome of claim 3, wherein the amount of the at least one fusogenic lipid is at least about 60% by weight of the lipid content of the gel or the liquid containing gel particles.

5. The liposome of claim 4, wherein the amount of the at least one fusogenic lipid is at least about 70% by weight of the lipid content of the gel or the liquid containing gel particles.

6. The liposome of claim 5, wherein the amount of the at least one fusogenic lipid is at least about 75% by weight of the lipid content of the gel or the liquid containing gel particles.

7. The liposome of claim 6, wherein the amount of the at least one fusogenic lipid is at least about 80% by weight of the lipid content of the gel or the liquid containing gel particles.

8. The liposome of claim 1, wherein the water-miscible organic solvent in step (A) or (B) is selected from the group consisting of acetaldehyde, acetone, acetonitrile, allyl alcohol, allylamine, 2-amino-1-butanol, 1-aminoethanol, 2-aminoethanol, 2-amino-2-ethyl-1,3-propanediol, 2-amino-2-methyl-1-propanol, 3-aminopentane, N-(3-aminopropyl)morpholine, benzylamine, bis(2-ethoxyethyl) ether, bis(2-hydroxyethyl) ether, bis(2-hydropropyl) ether, bis(2-methoxyethyl) ether, 2-bromoethanol, meso-2,3-butanediol, 2-(2-butoxyethoxy)-ethanol, butylamine, sec-butylamine, tert-butylamine, 4-butyrolacetone, 2-chloroethanol, 1-chloro-2-propanol, 2-cyanoethanol, 3-cyanopyridine, cyclohexylamine, diethylamine, diethylenetriamine, N,N-diethylformamide, 1,2-dihydroxy-4-methylbenzene, N,N-dimethylacetamide, N,N-dimethylformaide, 2,6-dimethylmorpholine, 1,4-dioxane, 1,3-dioxolane, dipentaerythritol, ethanol, 2,3-epoxy-1-propanol, 2-ethoxyethanol, 2-(2-ethoxyethoxy)-ethanol, 2-(2-ethoxyethoxy)-ethyl acetate, ethylamine, 2-(ethylamino)ethanol, ethylene glycol, ethylene oxide, ethylenimine, ethyl(−)-lactate, N-ethylmorpholine, ethyl-2-pyridine-carboxylate, formamide, furfuryl alcohol, furfurylamine, glutaric dialdehyde, glycerol, hexamethylphosphor-amide, 2,5-hexanedione, hydroxyacetone, 2-hydroxyethyl-hydrazine, N-(2-hydroxyethyl)-morpholine, 4-hydroxy-4-methyl-2-pentanone, 5-hydroxy-2-pentanone, 2-hydroxypropionitrile, 3-hydroxypropionitrile, 1-(2-hydroxy-1-propoxy)-2-propanol, isobutylamine, isopropylamine, 2-isopropylamino-ethanol, 2-mercaptoethanol, methanol, 3-methoxy-1-butanol, 2-methoxyethanol, 2-(2-methoxyethoxy)-ethanol, 1-methoxy-2-propanol, 2-(methylamino)-ethanol, 1-methylbutylamine, methylhydrazine, methyl hydroperoxide, 2-methylpyridine, 3-methylpyridine, 4-methylpyridine, N-methylpyrrolidine, N-methyl-2-pyrrolidinone, morpholine, nicotine, piperidine, 1,2-propanediol, 1,3-propanediol, 1-propanol, 2-propanol, propylamine, propyleneimine, 2-propyn-1-ol, pyridine, pyrimidine, pyrrolidine, 2-pyrrolidinone and quinoxaline.

9. The liposome of claim 8, wherein the organic solvent is methanol, ethanol, 1-propanol, 2-propanol, ethylene glycol or propylene glycol.

10. The liposome of claim 9, wherein the organic solvent is ethanol, 1-propanol or 2-propanol.

11. The liposome of claim 10, wherein the organic solvent is ethanol.

12. The liposome of claim 8, wherein the organic solvent is acetonitrile or acetone.

13. The liposome of claim 1, wherein aqueous medium Z1 and/or aqueous medium Z2 is an aqueous buffer.

14. The liposome of claim 1, wherein the gel or the liquid containing gel particles and aqueous medium Z1 are mixed by adding aqueous medium Z1 to the gel or the liquid.

15. The liposome of claim 1, wherein the gel or the liquid containing gel particles and aqueous medium Z1 are mixed by adding the gel or the liquid into aqueous medium Z1.

16. The liposome of claim 1, wherein the at least one nucleic acid is a DNA.

17. The liposome of claim 16, wherein the DNA is a plasmid DNA.

18. The liposome of claim 17, wherein the plasmid DNA is of up to about 20 kb in size.

19. The liposome of claim 18, wherein the DNA is a plasmid DNA of up to about 15 kb in size.

20. The liposome of claim 19, wherein the DNA is a plasmid DNA of up to about 10 kb in size.

21. The liposome of claim 18, wherein the DNA is a plasmid DNA of from about 0.5 kb to about 20 kb in size.

22. The liposome of claim 21, wherein the DNA is a plasmid DNA of from about 1 kb to about 15 kb in size.

23. The liposome of claim 22, wherein the DNA is a plasmid DNA of from about 2 kb to about 10 kb in size.

24. The liposome of claim 23, wherein the DNA is a plasmid DNA of from about 3 kb to about 7 kb in size.

25. The liposome of claim 1, wherein the at least one nucleic acid is an RNA.

26. The liposome of claim 25, wherein the RNA is an anti-sense RNA or RNA interference.

27. The liposome of claim 1, wherein the at least one nucleic acid is an oligonucleotide.

28. The liposome of claim 27, wherein the oligonucleotide is of about 5 to about 500 bases in size.

29. The liposome of claim 1, wherein the at least one liposome-forming lipid is selected from the group consisting of glycolipids, sphingolipids and phospholipids.

30. The liposome of claim 29, wherein the at least one liposome-forming lipid is selected from the group consisting of phospholipids.

31. The liposome of claim 30, wherein the at least one liposome-forming lipid is selected from the group consisting of phosphatidylcholine, phosphatidylserine, phosphatidylinositol, phosphatidylglycerol, diphosphatidylglycerol and N-acylphosphatidylethanolamine.

32. The liposome of claim 31, wherein the at least one liposome-forming lipid is selected from the group consisting of dioleoyl phosphatidylcholine, dipalmitoyl phosphatidylcholine, distearoyl phosphatidylcholine, dimyristoyl phosphatidylcholine, 1-palmitoyl-2-oleoyl-sn-glycero-3-phosphocholine, 1-oleoyl-1-palmitoyl-sn-glycero-3-phosphocholine, 1,2-dioleoyl-sn-glycero-3-[phospho-rac-(1-glycerol)], 1,2-dipalmitoyl-sn-glycero-3-[phospho-rac-(1-glycerol)], 1,2-distearoyl-sn-glycero-3-[phospho-rac-(1-glycerol)], 1,2-dimyristoyl-sn-glycero-3-[phospho-rac-(1-glycerol)], 1-palmitoyl-2-oleoyl-sn-glycero-3-[phospho-rac-(1-glycerol)], 1-oleoyl-2-palmitoyl-sn-glycero-3-[phospho-rac-(1-glycerol)], N-decanoyl phosphatidylethanolamine, N-dodecanoyl phosphatidylethanolamine and N-tetradecanoyl phosphatidylethanolamine.

33. The liposome of claim 1, further comprising a sterol.

34. The liposome of claim 33, wherein the sterol is cholesterol.

35. The liposome of claim 1, wherein the at least one fusogenic lipid is selected from the group consisting of N-acyl phosphatidylethanolamine.

36. The liposome of claim 35, wherein the N-acyl phosphatidylethanolamine is selected from the group consisting of N-decanoyl phosphatidylethanolamine, N-undecanoyl phosphatidylethanolamine, N-dodecanoyl phosphatidylethanolamine, N-tridecanoyl phosphatidylethanolamine and N-tetradecanoyl phosphatidylethanolamine.

37. The liposome of claim 36, wherein the N-acyl phosphatidylethanolamine. is 1,2-dioleoyl-sn-glycero-N-dodecanoyl-3-phosphoethanolamine.

38. The liposome of claim 1, wherein in the gel or the liquid containing gel particles a total amount of the at least one liposome-forming lipid and the at least one fusogenic lipid ranges from about 1% by weight of the gel or the liquid containing gel particles to the sum of the hydration limits of the at least one liposome-forming lipid and the at least one fusogenic lipid in water.

39. The liposome of claim 1, wherein in the gel or the liquid containing gel particles a total amount of the at least one liposome-forming lipid and the at least one fusogenic lipid ranges from about 5% to about 80% by weight of the gel or the liquid containing gel particles.

40. The liposome of claim 39, wherein said total amount ranges from about 10% to about 80% by weight of the gel or the liquid containing gel particles.

41. The liposome of claim 40, wherein said total amount ranges from about 15% to about 80% by weight of the gel or the liquid containing gel particles.

42. The liposome of claim 41, wherein said total amount ranges from about 20% to about 80% by weight of the gel or the liquid containing gel particles.

43. The liposome of claim 42, wherein said total amount ranges from about 30% to about 80% by weight of the gel or the liquid containing gel particles.

44. The liposome of claim 43, wherein said total amount ranges from about 40% to about 80% by weight of the gel or the liquid containing gel particles.

45. The liposome of claim 44, wherein said total amount ranges from about 50% to about 80% by weight of the gel or the liquid containing gel particles.

46. The liposome of claim 1, wherein in the gel or the liquid containing gel particles a total amount of the at least one liposome-forming lipid and the at least one fusogenic lipid ranges from about 10% to about 70% by weight of the gel or the liquid containing gel particles.

47. The liposome of claim 46, wherein said total amount ranges from about 20% to about 60% by weight of the gel or the liquid containing gel particles.

48. The liposome of claim 47, wherein said total amount ranges from about 30% to about 50% by weight of the gel or the liquid containing gel particles.

49. The liposome of claim 48, wherein said total amount is about 45% by weight of the gel or the liquid containing gel particles.

50. The liposome of claim 1, wherein aqueous medium Z1 is mixed in increments with the gel or the liquid containing gel particles, wherein the increments are up to about 100% of the weight of the gel or the liquid containing gel particles before the gel or the liquid is mixed with any aqueous medium Z1.

51. The liposome of claim 50, wherein the increments are up to about 80% of the weight of the gel or the liquid containing gel particles before the gel or the liquid is mixed with any aqueous medium Z1.

52. The liposome of claim 51, wherein the increments are up to about 60% of the weight of the gel or the liquid containing gel particles before the gel or the liquid is mixed with any aqueous medium Z1.

53. The liposome of claim 52, wherein the increments are up to about 40% of the weight of the gel or the liquid containing gel particles before the gel or the liquid is mixed with any aqueous medium Z1.

54. The liposome of claim 53, wherein the increments are up to about 20% of the weight of the gel or the liquid containing gel particles before the gel or the liquid is mixed with any aqueous medium Z1.

55. The liposome of claim 54, wherein the increments are up to about 10% of the weight of the gel or the liquid containing gel particles before the gel or the liquid is mixed with any aqueous medium Z1.

56. The liposome of claim 55, wherein the increments are up to about 5% of the weight of the gel or the liquid containing gel particles before the gel or the liquid is mixed with any aqueous medium Z1.

57. The liposome of claim 56, wherein the increments are up to about 1% of the weight of the gel or the liquid containing gel particles before the gel or the liquid is mixed with any aqueous medium Z1.

58. The liposome of claim 57, wherein the increments are up to about 0.5% of the weight of the gel or the liquid containing gel particles before the gel or the liquid is mixed with any aqueous medium Z1.

59. The liposome of claim 57, wherein the increments are up to about 0.1% of the weight of the gel or the liquid containing gel particles before the gel or the liquid is mixed with any aqueous medium Z1.

60. The liposome of claim 55, wherein the increments are from about 0.001% to about 10% of the weight of the gel or the liquid containing gel particles before the gel or the liquid is mixed with any aqueous medium Z1.

61. The liposome of claim 60, wherein the increments are from about 0.001% to about 5% of the weight of the gel or the liquid containing gel particles before the gel or the liquid is mixed with any aqueous medium Z1.

62. The liposome of claim 61, wherein the increments are from about 0.001% to about 1% of the weight of the gel or the liquid containing gel particles before the gel or the liquid is mixed with any aqueous medium Z1.

63. The liposome of claim 1, wherein the gel or the liquid containing gel particles comprises up to about 40 mg of the at least one nucleic acid per ml.

64. The liposome of claim 63, wherein the gel or the liquid containing gel particles comprises up to about 30 mg of the at least one nucleic acid per ml.

65. The liposome of claim 64, wherein the gel or the liquid containing gel particles comprises up to about 20 mg of the at least one nucleic acid per ml.

66. The liposome of claim 65, wherein the gel or the liquid containing gel particles comprises up to about 10 mg of the at least one nucleic acid per ml.

67. The liposome of claim 66, wherein the gel or the liquid containing gel particles comprises up to about 5 mg of the at least one nucleic acid per ml.

68. The liposome of claim 1, wherein the product of step (A), (B), (C), (D), (E), (F), (G) or (H) is washed with an aqueous medium by centrifugation, gel filtration or dialysis.

69. The liposome of claim 1, wherein the gel or the liquid containing gel particles is prepared by a method comprising the following steps:
(I) (a) (aa) mixing at least one liposome-forming lipid, the at least one fusogenic lipid, the at least one nucleic acid and a water-miscible organic solvent to form a mixture; or
(bb) (i) dissolving at least one liposome-forming lipid and the at least one fusogenic lipid in the water-miscible organic solvent to form an organic solution;
(ii) dissolving the at least one nucleic acid in aqueous medium X to form an aqueous solution; and
(iii) mixing the organic solution and aqueous solution to form a mixture; or
(b) mixing at least one liposome-forming lipid, the at least one fusogenic lipid and the water-miscible organic solvent to form a mixture; and thereafter
(II) (a) mixing the mixture of step (I)(a) with aqueous medium Y to form the gel or liquid containing gel particles; or
(b) mixing the mixture of step (I)(b) with the at least one nucleic acid and aqueous medium Y to form the gel or liquid containing gel particles,
wherein aqueous media X and Y are the same or different.

70. The liposome of claim 1, wherein the gel or the liquid containing gel particles is prepared by a method comprising the following steps:
(I) (a) (i) providing liposomes comprising the at least one liposome-forming lipid and the at least one fusogenic lipid, wherein the liposomes are prepared by a method other than the instant method; and
(ii) mixing the liposomes of step (1)(a)(i) with the at least one nucleic acid;
(b) (i) providing liposomes comprising the at least one liposome-forming lipid and the at least one fusogenic lipid in aqueous medium U, wherein the liposomes are prepared by a method other than the instant method; and
(ii) mixing the liposomes of step (I)(b)(i) with the at least one nucleic acid;
(c) (i) providing liposomes comprising the at least one liposome-forming lipid and the at least one fusogenic lipid, wherein the liposomes are prepared by a method other than the instant method; and
(ii) mixing the liposomes of step (I)(c)(i) with aqueous medium U and the at least one nucleic acid;
(d) (i) providing liposomes comprising the at least one liposome-forming lipid and the at least one fusogenic lipid in aqueous medium U, wherein the liposomes are prepared by a method other than the instant method; and
(ii) mixing the liposomes of step (I)(d)(i) with aqueous medium U and the at least one nucleic acid; or
(e) forming liposomes comprising the at least one liposome-forming lipid and the at least one fusogenic lipid in the presence of the at least one nucleic acid by a method other than the instant method;
(II) (a) mixing the product of step (I)(b), (I)(c) or (I)(d) with the water-miscible organic solvent to form the gel or the liquid containing gel particles; or
(b) mixing the product of step (I)(a) or (I)(e) with aqueous medium V and the water-miscible organic solvent to form the gel or the liquid containing gel particles,
wherein aqueous media U and V are the same or different.

71. The liposome of claim 1, wherein the gel or liquid containing gel particles does not contain any nucleic acid condensing agent and no nucleic acid condensing agent is used in step (A), (B), (C), (D), (E), (F), (G) or (H).

72. The liposome of claim 1, wherein the gel or liquid containing gel particles does not contain any hydrating agent and no hydrating agent is used in in step (A), (B), (C), (D), (E), (F), (G) or (H).

73. The liposome of claim 1, wherein a phospholipid content of the gel or the liquid containing gel particles is not 15 to 30% by weight of the gel or the liquid containing gel particles and the content of the water-miscible organic solvent is not 14 to 20% by weight of the gel or the liquid containing gel particles.

74. A method for preparing liposomes containing a nucleic acid encapsulated therein comprising the following steps:
(A) mixing a gel or a liquid containing gel particles with aqueous medium Z1 to directly form liposomes, wherein said gel or liquid containing gel particles comprises at least one liposome-forming lipid, at least one fusogenic lipid, a water-miscible organic solvent and the at least one nucleic acid, wherein the at least one liposome-forming lipid and the at least one fusogenic lipid are the same or different;
(B) (i) mixing a gel or a liquid containing gel particles with aqueous medium Z1 to form a curd or curdy substance, wherein said gel or liquid containing gel particles comprises at least one liposome-forming lipid, at least one fusogenic lipid, a water-miscible organic solvent and the at least one nucleic acid, wherein the at least one liposome-forming lipid and the at least one fusogenic lipid are the same or different; and
(ii) mixing the curd or curdy substance with aqueous medium Z2 to directly form the liposomes;
(C) (i) cooling a gel or a liquid containing gel particles to form a waxy substance, wherein said gel or liquid containing gel particles comprises at least one liposome-forming lipid, at least one fusogenic lipid, a water-miscible organic solvent and the at least one nucleic acid; and
(ii) mixing the waxy substance with aqueous medium Z1 to directly form the liposomes containing the at least one nucleic acid encapsulated therein;
(D) mixing a gel or a liquid containing gel particles with aqueous medium Z1 and the at least one nucleic acid to directly form the liposomes containing the at least one nucleic acid encapsulated therein, wherein said gel or liquid containing gel particles comprises at least one liposome-forming lipid, at least one fusogenic lipid and a water-miscible organic solvent;
(E) (i) mixing a gel or a liquid containing gel particles with aqueous medium Z1 and the at least one nucleic acid to form a curd or curdy substance, wherein said gel or liquid containing gel particles comprises at least one liposome-forming lipid, at least one fusogenic lipid and a water-miscible organic solvent; and
(ii) mixing the curd or curdy substance with aqueous medium Z2 to directly form the liposomes containing the at least one nucleic acid encapsulated therein,
(F) (i) mixing a gel or a liquid containing gel particles with aqueous medium Z1 to form a curd or curdy substance, wherein said gel or liquid containing gel particles comprises at least one liposome-forming lipid, at least one fusogenic lipid and a water-miscible organic solvent; and
(ii) mixing the curd or curdy substance with aqueous medium Z2 and the at least one nucleic acid to directly form the liposomes containing the at least one nucleic acid encapsulated therein;
(G) (i) cooling a gel or a liquid containing gel particles to form a waxy substance, wherein said gel or liquid containing gel particles comprises at least one liposome-forming lipid, at least one fusogenic lipid, a water-miscible organic solvent and the at least one nucleic acid; and
(ii) mixing the waxy substance with aqueous medium Z1 to directly form the liposomes containing the at least one nucleic acid encapsulated therein; or
(H) (i) cooling a gel or a liquid containing gel particles to form a waxy substance, wherein said gel or liquid containing gel particles comprises at least one liposome-forming lipid, at least one fusogenic lipid and a water-miscible organic solvent; and
(ii) mixing the waxy substance with aqueous medium Z1 and the at least one nucleic acid to directly form the liposomes containing the at least one nucleic acid encapsulated therein;
wherein the aqueous media Z1 and Z2 are the same or different and an amount of the at least one fusogenic lipid is at least 30% by weight of a lipid content of the gel or the liquid containing gel particles.

75. The method of claim 74, wherein the amount of the at least one fusogenic lipid is at least 40% by weight of a lipid content of the gel or the liquid containing gel particles.

76. The method of claim 75, wherein the amount of the at least one fusogenic lipid is at least 50% by weight of a lipid content of the gel or the liquid containing gel particles.

77. The method of claim 76, wherein the amount of the at least one fusogenic lipid is at least 60% by weight of a lipid content of the gel or the liquid containing gel particles.

78. The method of claim 77, wherein the amount of the at least one fusogenic lipid is at least 70% by weight of a lipid content of the gel or the liquid containing gel particles.

79. The method of claim 78, wherein the amount of the at least one fusogenic lipid is at least 75% by weight of a lipid content of the gel or the liquid containing gel particles.

80. The method of claim 79, wherein the amount of the at least one fusogenic lipid is at least 80% by weight of a lipid content of the gel or the liquid containing gel particles.

81. The method of claim 74, wherein the water-miscible organic solvent is selected from the group consisting of acetaldehyde, acetone, acetonitrile, allyl alcohol, allylamine, 2-amino-1-butanol, 1-aminoethanol, 2-aminoethanol, 2-amino-2-ethyl-1,3-propanediol, 2-amino-2-methyl-1-propanol, 3-aminopentane, N-(3-aminopropyl)morpholine, benzylamine, bis(2-ethoxyethyl) ether, bis(2hydroxyethyl) ether, bis(2-hydropropyl) ether, bis(2-methoxyethyl) ether, 2-bromoethanol, meso-2,3-butanediol, 2-(2-butoxyethoxy)-ethanol, butylamine, sec-butylamine, tert-butylamine, 4-butyrolacetone, 2-chloroethanol, 1-chloro-2-propanol, 2-cyanoethanol, 3-cyanopyridine, cyclohexylamine, diethylamine, diethylenetriamine, N,N-diethylformamide, 1,2-dihydroxy-4-methylbenzene, N,N-dimethylacetamide, N,N-dimethylformaide, 2,6-dimethylmorpholine, 1,4-dioxane, 1,3-dioxolane, dipentaerythritol, ethanol, 2,3-epoxy-1-propanol, 2-ethoxyethanol, 2-(2-ethoxyethoxy)-ethanol, 2-(2-ethoxyethoxy)-ethyl acetate, ethylamine, 2-(ethylamino)ethanol, ethylene glycol, ethylene oxide, ethylenimine, ethyl(−)-lactate, N-ethylmorpholine, ethyl-2-pyridine-carboxylate, formamide, furfuryl alcohol, furfurylamine, glutaric dialdehyde, glycerol, hexamethylphosphor-amide, 2,5-hexanedione, hydroxyacetone, 2-hydroxyethyl-hydrazine, N-(2-hydroxyethyl)-morpholine, 4-hydroxy-4-methyl-2-pentanone, 5-hydroxy-2-pentanone, 2-hydroxypropionitrile, 3-hydroxypropionitrile, 1-(2-hydroxy-1-propoxy)-2-propanol, isobutylamine, isopropylamine, 2-isopropylamino-ethanol, 2-mercaptoethanol, methanol, 3-methoxy-1-butanol, 2-methoxyethanol, 2-(2-methoxyethoxy)-ethanol, 1-methoxy-2-propanol, 2-(methylamino)-ethanol, 1-methylbutylamine, methylhydrazine, methyl hydroperoxide, 2-methylpyridine, 3-methylpyridine, 4-methylpyridine, N-methylpyrrolidine, N-methyl-2-pyrrolidinone, morpholine, nicotine, piperidine, 1,2-propanediol, 1,3-propanediol, 1-propanol, 2-propanol, propylamine, propyleneimine, 2-propyn-1-ol, pyridine, pyrimidine, pyrrolidine, 2-pyrrolidinone and quinoxaline.

82. The method of claim 81, wherein the organic solvent is acetonitrile, acetone or a C₁-C₃ alcohol.

83. The method of claim 82, wherein the organic solvent is methanol, ethanol, 1-propanol, 2-propanol, ethylene glycol or propylene glycol.

84. The method of claim 83, wherein the organic solvent is ethanol, 1-propanol or 2-propanol.

85. The method of claim 84, wherein the organic solvent is ethanol.

86. The method of claim 82, wherein the organic solvent is acetone.

87. The method of claim 74, wherein aqueous medium Z1 and/or aqueous medium Z2 is an aqueous buffer.

88. The method of claim 74, wherein the gel or the liquid containing gel particles and aqueous medium Z1 are mixed by adding aqueous medium Z1 to the gel or the liquid.

89. The method of claim 74, wherein the gel or the liquid containing gel particles and aqueous medium Z1 are mixed by adding the gel or the liquid into aqueous medium Z1.

90. The method of claim 74, wherein the at least one nucleic acid is a DNA.

91. The method of claim 90, wherein the DNA is a plasmid DNA.

92. The method of claim 91, wherein the plasmid DNA is of up to about 20 kb in size.

93. The method of claim 92, wherein the DNA is a plasmid DNA of up to about 15 kb in size.

94. The method of claim 93, wherein the DNA is a plasmid DNA of up to about 10 kb in size.

95. The method of claim 92, wherein the DNA is a plasmid DNA of from about 0.5 kb to about 20 kb in size.

96. The method of claim 95, wherein the DNA is a plasmid DNA of from about 1 kb to about 15 kb in size.

97. The method of claim 96, wherein the DNA is a plasmid DNA of from about 2 kb to about 10 kb in size.

98. The method of claim 97, wherein the DNA is a plasmid DNA of from about 3 kb to about 7 kb in size.

99. The method of claim 74, wherein the at least one nucleic acid is an RNA.

100. The method of claim 74, wherein the at least one nucleic acid is an oligonucleotide.

101. The method of claim 100, wherein the oligonucleotide is of about 5 to about 500 bases in size.

102. The method of claim 74, wherein the at least one liposome-forming lipid is selected from the group consisting of glycolipids, sphingolipids and phospholipids.

103. The method of claim 102, wherein the at least one liposome-forming lipid is selected from the group consisting of phospholipids.

104. The method of claim 103, wherein the at least one liposome-forming lipid is selected from the group consisting of phosphatidylcholine, phosphatidylserine, phosphatidylinositol, phosphatidylglycerol, diphosphatidylglycerol and N-acylphosphatidylethanolamine.

105. The method of claim 104, wherein the at least one liposome-forming lipid is selected from the group consisting of dioleoyl phosphatidylcholine, dipalmitoyl phosphatidylcholine, distearoyl phosphatidylcholine, dimyristoyl phosphatidylcholine, 1-palmitoyl-2-oleoyl-sn-glycero-3-phosphocholine, 1-oleoyl-2-palmitoyl-sn-glycero-3-phosphocholine, 1,2-dioleoyl-sn-glycero-3-[phospho-rac-(1-glycerol)], 1,2-dipalmitoyl-sn-glycero-3-[phospho-rac-(1-glycerol)], 1,2-distearoyl-sn-glycero-3-[phospho-rac-(1-glycerol)], 1,2-dimyristoyl-sn-glycero-3-[phospho-rac-(1-glycerol)], 1-palmitoyl-2-oleoyl-sn-glycero-3-[phospho-rac-(1-glycerol)], 1-oleoyl-2-palmitoyl-sn-glycero-3-[phospho-rac-(1-glycerol)], N-decanoyl phosphatidylethanolamine, N-dodecanoyl phosphatidylethanolamine and N-tetradecanoyl phosphatidylethanolamine.

106. The method of claim 74, further comprising adding a sterol in step (A), (B), (C), (D), (E), (F), (G) or (H).

107. The method of claim 106, wherein the sterol is cholesterol.

108. The method of claim 74, wherein the at least one fusogenic lipid is selected from the group consisting of N-acyl phosphatidylethanolamine.

109. The method of claim 108, wherein the N-acyl phosphatidylethanolamine is selected from the group consisting of N-decanoyl phosphatidylethanolamine, N-decanoyl phosphatidylethanolamine, N-undecanoyl phosphatidylethanolamine, N-tridecanoyl phosphatidylethanolamine and N-tetradecanoyl phosphatidylethanolamine.

110. The method of claim 109, wherein the N-acyl phosphatidylethanolamine. is 1,2-dioleoyl-sn-glycero-N-dodecanoyl-3-phosphoethanolamine.

111. The method of claim 74, wherein in the gel or the liquid containing gel particles a total amount of the at least one liposome-forming lipid and the at least one fusogenic lipid ranges from about 1% by weight of the gel or the liquid containing gel particles to the sum of the hydration limits of the at least one liposome-forming lipid and the at least one fusogenic lipid in water.

112. The method of claim 74, wherein in the gel or the liquid containing gel particles a total amount of the at least one liposome-forming lipid and the at least one fusogenic lipid ranges from about 5% to about 80% by weight of the the gel or the liquid containing gel particles.

113. The method of claim 112, wherein said total amount ranges from about 10% to about 80% by weight of the the gel or the liquid containing gel particles.

114. The method of claim 113, wherein said total amount ranges from about 15% to about 80% by weight of the the gel or the liquid containing gel particles.

115. The method of claim 114, wherein said total amount ranges from about 20% to about 80% by weight of the the gel or the liquid containing gel particles.

116. The method of claim 115, wherein said total amount ranges from about 30% to about 80% by weight of the the gel or the liquid containing gel particles.

117. The method of claim 116, wherein said total amount ranges from about 40% to about 80% by weight of the the gel or the liquid containing gel particles.

118. The method of claim 117, wherein said total amount ranges from about 50% to about 80% by weight of the the gel or the liquid containing gel particles.

119. The method of claim 118, wherein said total amount ranges from about 10% to about 70% by weight of the the gel or the liquid containing gel particles.

120. The method of claim 119, wherein said total amount ranges from about 20% to about 60% by weight of the the gel or the liquid containing gel particles.

121. The method of claim 120, wherein said total amount ranges from about 30% to about 50% by weight of the the gel or the liquid containing gel particles.

122. The method of claim 121, wherein said total amount is about 45% by weight of the the gel or the liquid containing gel particles.

123. The method of claim 74, wherein aqueous medium Z1 is mixed in increments with the gel or the liquid containing gel particles, wherein the increments are up to about 100% of the weight of the gel or the liquid containing gel particles before the gel or the liquid is mixed with any aqueous medium Z1.

124. The method of claim 123, wherein the increments are up to about 80% of the weight of the gel or the liquid containing gel particles before the gel or the liquid is mixed with any aqueous medium Z1.

125. The method of claim 124, wherein the increments are up to about 60% of the weight of the gel or the liquid containing gel particles before the gel or the liquid is mixed with any aqueous medium Z1.

126. The method of claim 125, wherein the increments are up to about 40% of the weight of the gel or the liquid containing gel particles before the gel or the liquid is mixed with any aqueous medium Z1.

127. The method of claim 126, wherein the increments are up to about 20% of the weight of the gel or the liquid containing gel particles before the gel or the liquid is mixed with any aqueous medium Z1.

128. The method of claim 127, wherein the increments are up to about 10% of the weight of the gel or the liquid containing gel particles before the gel or the liquid is mixed with any aqueous medium Z1.

129. The method of claim 128, wherein the increments are up to about 5% of the weight of the gel or the liquid containing gel particles before the gel or the liquid is mixed with any aqueous medium Z1.

130. The method of claim 129, wherein the increments are up to about 1% of the weight of the gel or the liquid containing gel particles before the gel or the liquid is mixed with any aqueous medium Z1.

131. The method of claim 130, wherein the increments are up to about 0.5% of the weight of the gel or the liquid containing gel particles before the gel or the liquid is mixed with any aqueous medium Z1.

132. The method of claim 131, wherein the increments are up to about 0.1% of the weight of the gel or the liquid containing gel particles before the gel or the liquid is mixed with any aqueous medium Z1.

133. The method of claim 128, wherein the increments are from about 0.001% to about 10% of the weight of the gel or the liquid containing gel particles before the gel or the liquid is mixed with any aqueous medium Z1.

134. The method of claim 133, wherein the increments are from about 0.001% to about 5% of the weight of the gel or the liquid containing gel particles before the gel or the liquid is mixed with any aqueous medium Z1.

135. The method of claim 134, wherein the increments are from about 0.001% to about 1% of the weight of the gel or the liquid containing gel particles before the gel or the liquid is mixed with any aqueous medium Z1.

136. The method of claim 74, wherein the gel or the liquid containing gel particles comprises up to about 40 mg of the at least one nucleic acid per ml.

137. The method of claim 136, wherein the gel or the liquid containing gel particles comprises up to about 30 mg of the at least one nucleic acid per ml.

138. The method of claim 137, wherein the gel or the liquid containing gel particles comprises up to about 20 mg of the at least one nucleic acid per ml.

139. The method of claim 138, wherein the gel or the liquid containing gel particles comprises up to about 10 mg of the at least one nucleic acid per ml.

140. The method of claim 139, wherein the gel or the liquid containing gel particles comprises up to about 5 mg of the at least one nucleic acid per ml.

141. The method of claim 74, wherein the product of step (A), (B), (C), (D), (E), (F), (G) or (H) is washed with an aqueous medium by centrifugation, gel filtration or dialysis.

142. The method of claim 74, wherein the gel or the liquid containing gel particles is prepared by a method comprising the following steps:
(I) (a) (aa) mixing at least one liposome-forming lipid, the at least one fusogenic lipid, the at least one nucleic acid and a water-miscible organic solvent to form a mixture; or
(bb) (i) dissolving at least one liposome-forming lipid and the at least one fusogenic lipid in the water-miscible organic solvent to form an organic solution;
(ii) dissolving the at least one nucleic acid in aqueous medium X to form an aqueous solution; and
(iii) mixing the organic solution and aqueous solution to form a mixture; or
(b) mixing at least one liposome-forming lipid, the at least one fusogenic lipid and the water-miscible organic solvent to form a mixture; and thereafter
(II) (a) mixing the mixture of step (I)(a) with aqueous medium Y and optionally the at least one nucleic acid to form the gel or liquid containing gel particles; or
(b) mixing the mixture of step (I)(b) with the at least one nucleic acid and aqueous medium Y to form the gel or liquid containing gel particles,
wherein aqueous media X and Y are the same or different.

143. The method of claim 74, wherein the gel or the liquid containing gel particles is prepared by a method comprising the following steps:
(I) (a) (i) providing liposomes comprising the at least one liposome-forming lipid and the at least one fusogenic lipid, wherein the liposomes are prepared by a method other than the instant method; and
(ii) mixing the liposomes of step (I)(a)(i) with the at least one nucleic acid;
(b) (i) providing liposomes comprising the at least one liposome-forming lipid and the at least one fusogenic lipid in aqueous medium U, wherein the liposomes are prepared by a method other than the instant method; and
(ii) mixing the liposomes of step (I)(b)(i) with the at least one nucleic acid;
(c) (i) providing liposomes comprising the at least one liposome-forming lipid and the at least one fusogenic lipid, wherein the liposomes are prepared by a method other than the instant method; and
(ii) mixing the liposomes of step (I)(c)(i) with aqueous medium U and the at least one nucleic acid;
(d) (i) providing liposomes comprising the at least one liposome-forming lipid and the at least one fusogenic lipid in aqueous medium U, wherein the liposomes are prepared by a method other than the instant method; and
(ii) mixing the liposomes of step (I)(d)(i) with aqueous medium U and the at least one nucleic acid;
(e) forming liposomes comprising the at least one liposome-forming lipid and the at least one fusogenic lipid in the presence of the at least one nucleic acid by a method other than the instant method; or
(f) forming liposomes comprising the at least one liposome-forming lipid and the at least one fusogenic lipid in aqueous medium U in the presence of the at least one nucleic acid by a method other than the instant method; and thereafter
(II) (a) mixing the product of step (I)(b), (I)(c), (I)(d) or (I)(f) with the water-miscible organic solvent to form the gel or the liquid containing gel particles; or
(b) mixing the product of step (I)(a) or (I)(e) with aqueous medium U and the water-miscible organic solvent to form the gel or the liquid containing gel particles,
wherein aqueous media U and V are the same or different.

144. The method of claim 74, wherein the gel or liquid containing gel particles does not contain any nucleic acid condensing agent and no nucleic acid condensing agent is used in step (A), (B), (C), (D), (E), (F), (G) or (H).

145. The method of claim 74, wherein no hydrating agent is used in in step (A), (B), (C), (D), (E), (F), (G) or (H) and wherein the gel or the liquid containing gel particles does not contain a hydrating agent.

146. The method of claim 74, wherein a phospholipid content of the gel or the liquid containing gel particles is not 15 to 30% by weight of the gel or the liquid and the content of the water-miscible organic solvent is not 14 to 20% by weight of the gel or the liquid containing gel particles.

147. A method for transfecting a eukaryotic cell with a plasmid DNA, comprising the following steps:
(a) providing the liposome of claim 1, wherein the at least one nucleic acid is the plasmid DNA; and thereafter
(b) contacting the cell with the liposome to transfect the cell with the plasmid DNA.

148. The method of claim 147, wherein step (b) is conducted by incubating the cell with the liposome at 37° C.

149. A method for transfecting a eukaryotic cell with a plasmid DNA in a eukaryotic subject in need of the transfection, said method comprising the following steps:
(a) providing the liposome of claim 1, wherein the at least one nucleic acid is the plasmid DNA; and thereafter
(b) administering the liposome in the eukaryotic subject.

150. The method of claim 149, wherein the liposome is administered intravenously in the eukaryotic subject.

151. The method of claim 150, wherein the eukaryotic subject is a human.

152. The method of claim 151, wherein the eukaryotic subject is a human in need of gene therapy and the plasmid DNA contains a gene necessary for the gene therapy.

153. The liposome of claim 1, wherein in the gel or the liquid containing gel particles a total amount of the at least one liposome-forming lipid and the at least one fusogenic lipid ranges from about 5% to about 95% by weight of the gel or the liquid containing gel particles.

154. The liposome of claim 153, wherein said total amount ranges from about 10% to about 95% by weight of the gel or the liquid containing gel particles.

155. The liposome of claim 154, wherein said total amount ranges from about 15% to about 95% by weight of the gel or the liquid containing gel particles.

156. The liposome of claim 155, wherein said total amount ranges from about 20% to about 95% by weight of the gel or the liquid containing gel particles.

157. The liposome of claim 156, wherein said total amount ranges from about 30% to about 95% by weight of the gel or the liquid containing gel particles.

158. The liposome of claim 157, wherein said total amount ranges from about 40% to about 95% by weight of the gel or the liquid containing gel particles.

159. The liposome of claim 158, wherein said total amount ranges from about 50% to about 95% by weight of the gel or the liquid containing gel particles.

160. The liposome of claim 1, wherein in the gel or the liquid containing gel particles a total amount of the at least one liposome-forming lipid and the at least one fusogenic lipid ranges from about 5% to about 90% by weight of the gel or the liquid containing gel particles.

161. The liposome of claim 160, wherein said total amount ranges from about 10% to about 90% by weight of the gel or the liquid containing gel particles.

162. The liposome of claim 160, wherein said total amount ranges from about 15% to about 90% by weight of the gel or the liquid containing gel particles.

163. The liposome of claim 162, wherein said total amount ranges from about 20% to about 90% by weight of the gel or the liquid containing gel particles.

164. The liposome of claim 163, wherein said total amount ranges from about 30% to about 90% by weight of the gel or the liquid containing gel particles.

165. The liposome of claim 164, wherein said total amount ranges from about 40% to about 90% by weight of the gel or the liquid containing gel particles.

166. The liposome of claim 165, wherein said total amount ranges from about 50% to about 90% by weight of the gel or the liquid containing gel particles.

167. The liposome of claim 166, wherein said total amount ranges from about 60% to about 90% by weight of the gel or the liquid containing gel particles.

168. The liposome of claim 73, wherein the content of the water-miscible organic solvent is not 14 to 20% by weight of the gel or the liquid.
